(19) United States
(12) Patent Application Publication
(10) Pub. No.: US 2012/0135425 A1
(43) Pub. Date: May 31, 2012

(54) ELISA for Haptoglobin-Matrix Metalloproteinase 9 Complex as a Diagnostic Test for Conditions Including Acute Inflammation

(75) Inventors: Jeffrey Lakritz, Columbus, OH (US); Grigori Andreevich Bannikov, Dublin, OH (US); Tatiana Karelina, Dublin, OH (US)

(73) Assignee: THE OHIO STATE UNIVERSITY RESEARCH FOUNDATION, Columbus, OH (US)

(21) Appl. No.: 13/215,686

(22) Filed: Aug. 23, 2011

Related U.S. Application Data
(63) Continuation-in-part of application No. 12/861,395, filed on Aug. 23, 2010.
(60) Provisional application No. 61/472,815, filed on Apr. 7, 2011.

(51) Int. Cl. G01N 33/573; G01N 21/64

(52) U.S. Cl. 435/7.92; 435/7.1; 435/7.9

(57) ABSTRACT

A method for detecting a haptoglobin-matrix metalloproteinase 9 (Hp-MMP 9) complex in a biological sample. The sample includes incubating the biological sample with a capture reagent immobilized on a solid support to bind Hp-MMP 9 to the capture reagent. The capture reagent includes a monoclonal antibody that binds MMP9. The method detects Hp-MMP 9 bound to the immobilized capture reagent by contacting the bound Hp-MMP 9 with a detectable antibody that binds to Hp.

### CROSS-REFERENCE TO RELATED APPLICATIONS

This application is a continuation-in-part of U.S. patent application Ser. No. 12/861,395, entitled “ELISA for Haptoglobin-Matrix Metalloproteinase 9 Complex as a Diagnostic Test for Conditions Including Acute Inflammation,” filed on Aug. 23, 2010, and this application claims the benefit of U.S. Patent Application Ser. No. 61/472,815, entitled “ELISA for Haptoglobin-Matrix Metalloproteinase 9 Complex as a Diagnostic Test for Conditions Including Acute Inflammation,” filed on Apr. 7, 2011, the disclosures of which are incorporated by reference herein in their entireties.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant Agreement No. 2008-35204-04473 awarded by the National Institute of Food and Agriculture.

### FIELD OF THE INVENTION

The present invention relates to methods of diagnosing or predicting acute inflammation and conditions including acute inflammation in a mammal.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Early detection of a disease condition typically allows for a more effective therapeutic treatment with a correspondingly more favorable clinical outcome. In many cases, however, early detection of disease symptoms is problematic; hence, a disease may become relatively advanced before diagnosis is possible. Systemic inflammatory conditions represent one such class of disease symptoms. These conditions present in humans and other mammals.

Systemic inflammatory conditions, e.g., sepsis, may result from an interaction between a pathogenic microorganism and the host's defense system that triggers an excessive and dysregulated inflammatory response in the host. The complexity of the host's response during the systemic inflammatory response may complicate efforts towards understanding disease pathogenesis. And, this incomplete understanding of the disease pathogenesis, in turn, contributes to the difficulty in finding diagnostic biomarkers. Early and reliable diagnosis is imperative, however, because of the remarkably rapid progression of sepsis into a life-threatening condition.

Systemic inflammatory conditions follow a well-described time course, progressing from systemic inflammatory response syndrome (“SIRS”) -negative to SIRS-positive to sepsis, which may progress to severe sepsis, septic shock, multiple organ failure (“MOF”), and ultimately death. Sepsis also may arise in an infected individual when the individual subsequently develops SIRS. “SIRS” is often defined by the presence of two or more of the following parameters: a body temperature greater than 38° C. or less than 36° C.; a heart rate greater than 90 beats per minute; a respiratory rate greater than 20 breaths per minute; P_{CO2} less than 32 mm Hg; and a white blood cell count either less than 4.0×10⁹ cells/L or greater than 12.0×10⁹ cells/L, or having greater than 10% immature band forms. “Sepsis” is commonly defined as SIRS coupled with a confirmed infectious process. “Severe sepsis” is associated with MOF, hypotension, disseminated intravascular coagulation (“DIC”), or hypoperfusion abnormalities, including lactic acidosis, oliguria, and changes in mental status. “Septic shock” is commonly defined as sepsis-induced hypotension that is resistant to fluid resuscitation with the additional presence of hypoperfusion abnormalities.

Apart from such conditions in humans, the presentation of systemic inflammatory conditions in other mammals can have adverse consequences as well. For example, bovine respiratory disease (BRD) and other acute diseases continue to have a major impact on livestock productivity in the United States. Recent studies by the National Animal Health Monitoring System (“NAHMS”) suggest BRD is a leading cause of illness and death in U.S. feedlots. Under field conditions, the use of acute phase proteins (APP) to detect animals requiring treatment and animals developing lung lesions has demonstrated some utility. For example, haptoglobin (Hp) responses to inflammation in cattle have been evaluated in acute bronchopneumonia, [Eckersall P D, Young F J, McComb C, Hogarth C J, Safi S, Weber A, McDonald T, Nolan A M, Fitzpatrick J L (2001), Acute phase proteins in serum and milk from dairy cows with clinical mastitis, Vet. Rec. 148: 35-41; Humblet M F, Coghe J, Lekeux P, Godeau J M (2004), Acute phase proteins assessment for an early selection of treatments in growing calves suffering from bronchopneumonia under field conditions, Res. Vet. Sci. 77: 41-4; Katoh N, Miyamoto T, Nakagawa H, Watanabe A (1999), Detection of annexin I and IV and haptoglobin in bronchoalveolar lavage fluid from calves experimentally inoculated with Pasteurella haemolytica, Am. J. Vet. Res. 60: 1390-1395; Morimatsu M, Syuto B, Shimada N, Fujinaga T, Yamamoto S, Saito M, Naiki M (1991), Isolation and characterization of bovine haptoglobin from acute phase sera, J. Biol. Chem. 266: 11833-11837; Wittum T E, Young C R, Stanker L H, Griffin D D, Perino L J, Littledike E T (1996), Haptoglobin response to clinical respiratory tract disease in feedlot cattle, Am. J. Vet. Res. 57: 646-649], acute rumen acidosis [Gozho G N, Plaizier J C, Krause D O, Kennedy A D, Wittenberg K M (2005), Subacute ruminal acidosis induces ruminal lipopolysaccharide endotoxin release and triggers an inflammatory response, J. Dairy Sci. 88:1399-1403], coliform mastitis [Nielsen B H, Jacobsen S, Andersen P H, Niewold T A, Heegaard P M (2004), Acute phase protein concentrations in serum and milk from healthy cows, cows with clinical mastitis and cows with extramammary inflammatory conditions, Vet. Rec. 154:361-365; Ohtsuka H, Kudo K, Mori K, Nagai F, Hatsugaya A, Tajima M, Tamura K, Hoshi F, Koiwa M, Kawamura S (2001), Acute phase response in naturally occurring coliform mastitis, J. Vet. Med. Sci. 63:675-678], hepatic lipidosis, [Katoh N, Nakagawa H (1999), Detection of haptoglobin in the high-density lipoprotein and the very high-density lipoprotein fractions from sera of calves with experimental pneumonia and cows with naturally occurring fatty liver, J. Vet. Med. Sci. 61:503 119-124; Stengarde L, Traven M, Emanuelson U, Holtenius K, Hultgren J, Niskanen R (2008), Metabolic profiles in five high-producing Swedish dairy herds with a history of abomasal displacement and ketosis, Acta Vet. Scand. 50:31], and transport stress [Arthington J D, Eichert S D, Kunkle W E, Martin F G (2003), Effect of transportation and commingling on the acute-phase protein response, growth, and feed intake of newly weaned beef calves, J. Anim Sci. 81:1120-1125; Murata H, Miyamoto T (1993), Bovine haptoglobin as a possible immunomodulator in the sera of transported calves, Br. Vet. J. 149:277-283].

Serum concentrations of Hp in acutely ill cattle increase (>100 fold), reaching maximum concentrations between 48-96 h [Eckersall P D, Young F J, McComb C, Hogarth C J, Safi S, Weber A, McDonald T, Nolan A M, Fitzpatrick J L (2001), Acute phase proteins in serum and milk from dairy cows with clinical mastitis, Vet. Rec. 148:35-41; Hiss S, Mielenz M, Bruckmaier R M, Sauerwein H (2004), Haptoglobin concentrations in blood and milk after endotoxin challenge and quantification of mammary Hp mRNA expression, J. Dairy Sci. 87:3778-3784; Katoh N, Miyamoto T, Nakagawa H, Watanabe A (1999), Detection of annexin I and IV and haptoglobin in bronchoalveolar lavage fluid from calves experimentally inoculated with Pasteurella haemolytica, Am. J. Vet. Res. 60:1390-1395; Larsen K, Macleod D, Nihlberg K, Gurcan E, Bjermer L, Marko-Varga G, Westergren-Thorsson G (2006), Specific haptoglobin expression in bronchoalveolar lavage during differentiation of circulating fibroblast progenitor cells in mild asthma, J. Proteome. Res. 5:1479-1483; Morimatsu M, Syuto B, Shimada N, Fujinaga T, Yamamoto S, Saito M, Naiki M (1991), Isolation and characterization of bovine haptoglobin from acute phase sera, J. Biol. Chem. 266:11833-11837; Tseng C F, Lin C C, Huang H Y, 556 Liu H C, Mao S J (2004), Antioxidant role of human haptoglobin, Proteomics. 4:2221-2228]. However, the reliability of serum Hp responses as indicators of morbidity are less useful than correlations of its reduction with appropriate treatment and clinical resolution of disease in calves with BRD [Berry B A, Confer A W, Krehbiel C R, Gill D R, Smith R A, Montelongo M (2004), Effects of dietary energy and starch concentrations for newly received feedlot calves: II. Acute phase protein response, J. Anim Sci. 82:845-850; Humblet M F, Coghe J, Lekeux P, Godeau J M (2004), Acute phase proteins assessment for an early selection of treatments in growing calves suffering from bronchopneumonia under field conditions, Res. Vet. Sci. 77:41-47]. Observed moderate increases of serum Hp are described for cows with some chronic illnesses, which do not necessarily have apparent signs of inflammation [Gronlund U, Hallen S C, Persson 489 WK (2005), Haptoglobin and serum amyloid A in milk from dairy cows with chronic sub-clinical mastitis, Vet. Res. 36:191-198; Nakagawa H, Yamamoto O, Oikawa S, Higuchi H, Watanabe A, Katoh N (1997), Detection of serum haptoglobin by enzyme-linked immunosorbent assay in cows with fatty liver, Res. Vet. Sci. 62:137-141], and this diminishes this test's value as an indicator of inflammation.

And, as described above, current methods of diagnosis of acute inflammation, systemic infection, neutrophil activation and sepsis in humans also have numerous limitations, and so a new, rapid, and reliable test could improve treatment and outcomes [Mancini N, Carletti S, Ghidoli N, Cichero 522 P, Burioni R, Clementi M (2010), The era of molecular and other non-culture-based methods in diagnosis of sepsis, Clin. Microbiol. Rev. 23:235-251].

A need, therefore, exists for a method of diagnosing sepsis (and other conditions including acute inflammation) in humans and other mammals sufficiently early to allow effective intervention and prevention. Most existing sepsis scoring systems or predictive models predict only the risk of late-stage complications, including death, in patients who already are considered septic. Such systems and models, however, do not predict the development of sepsis itself. What is further needed is a method to predict acute inflammation in humans that are not presenting symptoms and other mammals that are not presenting signs. Generally, researchers will define a biomarker or biomarkers expressed at a different level in a group of septic patients versus a normal (i.e., non-septic) control group of patients. U.S. Pat. No. 7,465,555, discloses a method of indicating early sepsis by analyzing time-dependent changes in the expression level of various biomarkers. Accordingly, optimal methods of diagnosing early sepsis currently require both measuring a plurality of biomarkers and monitoring the expression of these biomarkers over a period of time.

There is a continuing urgent need in the art to diagnose sepsis with specificity and sensitivity, without the need for monitoring a subject over time. Ideally, diagnosis would be made by a technique that accurately and rapidly measures a single biomarker at a single point in time, thereby allowing for early diagnosis and minimizing disease progression during the time required for diagnosis.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As described above, there presently is no reliable and efficient method to detect if a subject (e.g., human or other mammal) is suffering from an acute inflammatory condition or at risk of same. Ideally, diagnosis would be made by a technique that accurately and rapidly measures a single biomarker at a single point in time, thereby allowing for early diagnosis and minimizing disease progression during the time required for diagnosis.

Previously, covalent, heteromeric complexes of Hp and matrix metalloproteinase 9 (MMP 9) have been identified in neutrophil granules and the serum of cattle with acute septic inflammation of the abdomen or thorax [Bannikov G A, Mattoon J S, Abrahamsen E J, Premanandan C, Green-Church K B, Marsh A E, Lakritz J (2007), Biochemical and enzymatic characterization of purified covalent complexes of matrix metalloproteinase-9 and haptoglobin released by bovine granulocytes in vitro, Am. J. Vet. Res. 68:995-1004]. In most of these cases, mixed bacterial sepsis was evident [Bannikov G A, Mattoon J S, Abrahamsen E J, Premanandan C, Green-Church K B, Marsh A E, Lakritz J (2007), Biochemical and enzymatic characterization of purified covalent complexes of matrix metalloproteinase-9 and haptoglobin released by bovine granulocytes in vitro, Am. J. Vet. Res. 68:995-1004]. In contrast to free Hp which is produced mainly by the liver during inflammation, Hp-MMP 9 complexes are released from neutrophils upon degranulation [Bannikov G A, Mattoon J S, Abrahamsen E J, Premanandan C, Green-Church K B, Marsh A E, Lakritz J (2007), Biochemical and enzymatic characterization of purified covalent complexes of matrix metalloproteinase-9 and haptoglobin released by bovine granulocytes in vitro, Am. J. Vet. Res. 68:995-1004]. This suggests that Hp-MMP 9 complexes present in serum are a manifestation of neutrophil activation and degranulation.

Various aspects of the present invention are directed to the concept that complexes of haptoglobin (Hp) and matrix metalloproteinase 9 (MMP 9), i.e., Hp-MMP 9 complexes, produced by neutrophils in vitro and found in acute phase sera, have specific functional significance that differs from un-complexed forms of free Hp and MMP 9 alone. Thus, serum concentrations of Hp-MMP 9 may serve as an independent indicator of clinically important events occurring during acute inflammation. As such, there is a utility of an Hp-MMP 9 complex ELISA in comparison to ELISA for un-complexed Hp or MMP 9 alone as an indicator of acute septic inflammatory disease in mammals, by providing a method to specifically identify systemic neutrophil activation in humans, cattle, and other mammals.

Thus, one aspect of the present invention provides a method of detecting whether a subject is suffering from an acute inflammatory condition or at risk of same by detecting the presence of a biomarker, or a particular concentration of a biomarker, in a sample from the subject. The sample may be a blood sample, such as the serum fraction. In one embodiment, the biomarker may include a protein, such as a serum protein. The biomarker may include the serum protein haptoglobin, or isoforms of the serum protein haptoglobin. In one embodiment, the biomarker may include a protein, such as a serum protein, in complex with an enzyme, such as a matrix metalloproteinase, such as matrix metalloproteinase 9 (an Hp-MMP 9 complex). The biomarker may include various isoforms of the Hp-MMP 9 complex. In one embodiment, the method may include determining the concentration of the biomarker in samples collected from a subject. The method of this embodiment may also include comparing that concentration to a range of standard concentrations. In another embodiment, the method may include comparing the level of the biomarker in samples collected from the subject at different times. In yet another embodiment, the method may include comparing the biomarker with a reference biomarker from the same subject or a different subject.

Another aspect of the invention provides a test for performing the method described above. In one embodiment, the test may be an enzyme-linked immunosorbent assay (ELISA).

One such ELISA test embodiment is directed to a method for detecting one or more isoforms of an Hp-MMP 9 complex in a biological sample. The method includes: (1) incubating a biological sample with a capture reagent immobilized on a solid support to bind multiple isoforms of Hp-MMP 9 to the capture reagent, wherein the capture reagent includes an antibody, such as a monoclonal antibody, that binds MMP9; and (2) detecting Hp-MMP 9 bound to the immobilized capture reagent by contacting the bound Hp-MMP 9 with a detectable antibody that binds to Hp.

Another ELISA test embodiment is directed to a process for identifying a patient with an acute inflammatory condition or at risk of an acute inflammatory condition by determining concentration of Hp-MMP 9 in a bodily fluid sample. The process includes (a) obtaining monoclonal antibodies specific for MMP9 and attaching the monoclonal antibodies to a solid support; (b) obtaining a sample of a bodily fluid from a patient wherein the sample is suspected of containing Hp-MMP 9 and/or immunogenic fragments of Hp-MMP 9; (c) adding the sample to the monoclonal antibodies of step (a) wherein the Hp-MMP 9 and/or immunogenic fragments of Hp-MMP 9 contained in the sample is captured by the monoclonal antibodies; (d) providing second antibodies specific for Hp; (e) labeling the second antibodies with a detector; (f) adding the second antibodies of step (e) to the Hp-MMP 9 and/or immunogenic fragments of Hp-MMP 9 captured in step (c) wherein the second antibodies of step (e) bind to the Hp-MMP 9 and/or immunogenic fragments of Hp-MMP 9 captured in step (c); (g) adding a reporter that reacts with the detector to form a reaction product; and (h) measuring the reaction product to determine concentration of Hp-MMP 9 in the sample; and (i) determining if the concentration of Hp-MMP 9 in the sample is elevated above a selected cut-off concentration indicative of concentration of Hp-MMP 9, wherein the elevated concentration identifies that the patient has an acute inflammatory condition or is at risk of an acute inflammatory condition.

Another ELISA test embodiment is directed to a method for detecting Hp-MMP 9 in plasma or serum to screen for an acute inflammatory condition or risk of same. This test includes the steps of: (a) providing polyclonal or monoclonal antibodies against MMP9; (b) providing a microtiter plate coated with the antibodies; (c) adding the serum or plasma to the microtiter plate; (d) providing horseradish peroxidase-anti-Hp conjugates reactive with Hp to the microtiter plate; (e) providing hydrogen peroxide as a reactor to the microtiter plate; and (f) comparing the reaction which occurs as a result of steps (a) to (e) with a standard curve to determine the level of Hp-MMP 9 compared to a normal individual.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a graphical representation of ELISA data demonstrating the specificity of the MMP 9 and Hp-MMP 9 ELISA assays for free MMP 9 and Hp-MMP 9 complexes. The graph is representative of results of 3 independent experiments. The line with solid black circles (--) represents absorbance for increasing concentrations of Hp-MMP 9 captured on MMP 9 monoclonal Ab and detected with anti-Hp HRP conjugate (Hp-MMP 9 standard curve). The line with open circles (-∘-) represents wells where affinity purified Hp was added to the anti-MMP 9 coated wells followed by anti-Hp HRP conjugate. The line having open, downward triangles represents wells where affinity purified MMP 9 was added to wells followed by anti-Hp HRP conjugate. The line with open, upward triangles (-Δ-) represents wells where Hp-MMP 9 complexes were added to wells, and HRP-conjugated anti-MMP 9 (clone 10.1) was added to wells (simulation of MMP 9 ELISA).

FIG. 2A is a plot of data for serum haptoglobin in cattle by disease classification (where disease classification “1”=acute septic disease, “2”=chronic or metabolic disease, and “3”=normal) and box plot overlay. Open upward triangles (Δ) represent acute septic animals, downward triangles represent chronic/metabolic disease, and diamonds (⋄) represent normal animals. Box plots depict the median (solid line), mean (dashed line), 5th percentile, 25th percentile, 75th percentile and 95th percentile. Filled triangles or diamonds depict outliers.

FIG. 2B is a plot of data for serum Hp-MMP 9 in cattle by disease classification (where disease classification “1”=acute septic disease, “2”=chronic or metabolic disease, and “3”=normal) and box plot overlay. Open upward triangles (Δ) represent acute septic animals, downward triangles represent chronic/metabolic disease, and diamonds (⋄) represent normal animals. Box plots depict the median (solid line), mean (dashed green line), 5th percentile, 25th percentile, 75th percentile and 95th percentile. Filled triangles or diamonds depict outliers.

FIG. 2C is a plot of data for serum MMP9 in cattle by disease classification (where disease classification “1”=acute septic disease, “2”=chronic or metabolic disease, and “3”=normal) and box plot overlay. Open upward triangles (Δ) represent acute septic animals, downward triangles represent chronic/metabolic disease, and diamonds (⋄) represent normal animals. Box plots depict the median (solid line), mean (dashed green line), 5th percentile, 25th percentile, 75th percentile and 95th percentile. Filled triangles or diamonds depict outliers.

FIG. 2D shows the box plot data from FIG. 2A. The box plot depicts the median (solid line), mean (dashed line), 5th percentile (lower end/cap bar), 25th percentile (bottom of box), 75th percentile (top of box) and 95th percentile (upper end/cap bar). Filled triangles or diamonds depict outliers.

FIG. 2E shows the box plot data from FIG. 2B. The box plot depicts the median (solid line), mean (dashed line), 5th percentile (lower end/cap bar), 25th percentile (bottom of box), 75th percentile (top of box) and 95th percentile (upper end/cap bar). Filled triangles or diamonds depict outliers.

FIG. 2F shows the box plot data from FIG. 2C. The box plot depicts the median (solid line), mean (dashed line), 5th percentile (lower end/cap bar), 25th percentile (bottom of box), 75th percentile (top of box) and 95th percentile (upper end/cap bar). Filled triangles or diamonds depict outliers.

FIG. 3 is a graph showing the effect of lipopolysaccharide (LPS) infusion on Hp-MMP 9 and total white blood cell (WBC) count in cattle.

FIG. 4 is a graph showing the effect of LPS infusion on Hp-MMP 9 and serum cortisol in cattle.

FIG. 5 is a graph showing the effect of LPS infusion on Hp-MMP 9 and band PMN (polymorphonuclears) in cattle.

FIG. 6 is a graph showing the effect of LPS infusion on serum Hp-MMP 9 and Hp in cattle.

FIG. 7 is a graph showing the effect of LPS infusion on Hp-MMP 9 and serum α1-acid glycoprotein (AGP) in cattle.

FIG. 8 is a graph showing serum Hp-MMP 9 in experimental Mycoplasma bovis infection.

FIG. 9 is a graph showing the presence of serum Hp-MMP 9 in human patients having systemic lupus erythematosis, rheumatoid arthritis, or osteoarthritis.

FIG. 10 is a graph showing various white blood cell counts post injection of LPS for a first calf (calf 101).

FIG. 11 is a graph showing various white blood cell counts post injection of LPS for a second calf (calf 102).

FIG. 12 is a graph showing various white blood cell counts post injection of LPS for a third calf (calf 104).

FIG. 13 is a graph showing concentrations of neutrophils and Hp-MMP 9 in the calf of FIG. 10 (calf 101) post injection of LPS.

FIG. 14 is a graph showing concentrations of neutrophils and Hp-MMP 9 in the calf of FIG. 11 (calf 102) post injection of LPS.

FIG. 15 is a graph showing concentrations of neutrophils and Hp-MMP 9 in the calf of FIG. 12 (calf 104) post injection of LPS.

FIG. 16 is a graph showing the effect of a bacterial challenge (i.e., bacterial inoculation of calves) on serum Hp-MMP 9 complexes and Hp in beef calves (with circles showing Hp-MMP 9 in calves challenged with the bacteria, triangles showing Hp-MMP 9 in control calves, squares showing Hp in calves challenged with the bacteria, and diamonds showing Hp in control calves).

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, there presently is no reliable and efficient method to detect if a subject is suffering from an acute inflammatory condition or at risk of same.

Definitions

The term “Hp-MMP 9” as used herein refers to a complex of haptoglobin and matrix metalloproteinase 9. A complex is a group of two or more associated polypeptide chains—and herein the Hp-MMP 9 complex is a group of two associated polypeptide chains: the chain for haptoglobin and the chain for matrix metalloproteinase 9. As is known to those of ordinary skill in the art, haptoglobin is a protein that in humans is encoded by the Hp gene. In blood plasma, haptoglobin binds free hemoglobin (Hb) released from erythrocytes with high affinity and thereby inhibits its oxidative activity. The haptoglobin-hemoglobin complex will then be removed by the reticuloendothelial system (mostly the spleen). Matrix metalloproteinases (MMPs) are zinc-dependent endopeptidases. They are capable of degrading all kinds of extracellular matrix proteins, but also can process a number of bioactive molecules. They are known to be involved in the cleavage of cell surface receptors, the release of apoptotic ligands (such as the FAS ligand), and chemokine/cytokine in/activation. MMPs are also thought to play a major role on cell behaviors such as cell proliferation, migration (adhesion/dispersion), differentiation, angiogenesis, apoptosis and host defense. The MMPs share a common domain structure. The three common domains are the pro-peptide, the catalytic domain and the haemopexin-like C-terminal domain which is linked to the catalytic domain by a flexible hinge region. Of the MMPs, two—MMP2 and MMP9—are gelatinases. The main substrates of the gelatinases are type IV collagen and gelatin, and these enzymes are distinguished by the presence of an additional gelatin-binding domain inserted into the catalytic domain. This gelatin-binding region is positioned immediately before the zinc binding motif, and forms a separate folding unit which does not disrupt the structure of the catalytic domain.

The term “detecting” is used in the broadest sense to include both qualitative and quantitative measurements of a target molecule. In one aspect, the detecting method as described herein is used to identify the mere presence of Hp-MMP 9 in a biological sample. In another aspect, the method is used to test whether Hp-MMP 9 in a sample is at a particular level. In yet another aspect, the method can be used to quantify the amount of Hp-MMP 9 in a sample and further to compare the Hp-MMP 9 levels from different samples or compare the amount of Hp-MMP 9 in a sample to reference standards.

The term “biological sample” refers to a body sample from any animal, such as any mammal, such as a human. Such samples include biological fluids such as serum, plasma, vitreous fluid, lymph fluid, synovial fluid, follicular fluid, seminal fluid, amniotic fluid, milk, whole blood, urine, cerebro-spinal fluid, saliva, sputum, lung lavage, tears, perspiration, mucus, and tissue culture medium, as well as tissue extracts such as homogenized tissue, and cellular extracts.

The term “capture reagent” refers to a reagent capable of binding and capturing a target molecule in a sample such that under suitable condition, the capture reagent-target molecule complex can be separated from the rest of the sample. Typically, the capture reagent is immobilized or immobilizable. In a sandwich immunoassay, the capture reagent may be an antibody or a mixture of different antibodies against a target antigen.

The term “detectable antibody” refers to an antibody that is capable of being detected either directly through a label amplified by a detection agent, or indirectly through, e.g., another antibody that is labeled. For direct labeling, the antibody is typically conjugated to a moiety that is detectable by some means. One such antibody is an antibody conjugated to horse radish peroxidase.

The term “detection agent” refers to a moiety or technique used to detect the presence of the detectable antibody, and includes detection agents that amplify the immobilized label such as a label captured onto a microtiter plate. On such detection agent is hydrogen peroxide.

The term “antibody” is used in the broadest sense and includes monoclonal antibodies (including agonist, antagonist, and neutralizing antibodies), polyclonal antibodies, multivalent antibodies, multispecific antibodies, and antibody fragments so long as they exhibit the desired binding specificity.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic epitope. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method [described by Kohler et al. Nature 256:495 (1975)], or may be made by recombinant DNA methods (see, e.g., U.S. Pat. No. 4,816,567). The “monoclonal antibodies” may also be isolated from phage antibody libraries using the techniques described in Clackson et al. Nature 352:624-628 (1991) and Marks et al. J. Mol. Biol. 222:581-597 (1991), for example.

“Mammal” for purposes of treatment refers to any animal classified as a mammal, including humans, domestic, and farm animals, and zoo, sports, or pet animals, such as dogs, horses, cats, sheep, pigs, cows, etc. In various aspects of the present invention, the mammal may be human or cattle.

“Systemic inflammatory response syndrome,” or “SIRS,” refers to a clinical response to a variety of severe clinical insults, as manifested by two or more of the following conditions within a 24-hour period in a human: body temperature greater than 38° C. (100.4° F.) or less than 36° C. (96.8° F.); heart rate (HR) greater than 90 beats/minute; respiratory rate (RR) greater than 20 breaths/minute, or P_{CO2} less than 32 mm Hg, or requiring mechanical ventilation; and white blood cell count (WBC) either greater than 12.0×10⁹/L or less than 4.0×10⁹/L or having greater than 10% immature forms (bands).

These symptoms of SIRS represent a consensus definition of SIRS that may be modified or supplanted by an improved definition in the future. The present definition is used to clarify current clinical practice and does not represent a critical aspect of the invention.

A subject with SIRS has a clinical presentation that is classified as SIRS, as defined above, but is not clinically deemed to be septic.

“Sepsis” refers to a SIRS-positive condition that is associated with a confirmed infectious process. Clinical suspicion of sepsis arises from the suspicion that the SIRS-positive condition of a SIRS patient is a result of an infectious process. As used herein, “sepsis” includes all stages of sepsis including, but not limited to, the onset of sepsis, severe sepsis and MOF associated with the end stages of sepsis.

The “onset of sepsis” refers to an early stage of sepsis, i.e., prior to a stage when the clinical manifestations are sufficient to support a clinical suspicion of sepsis. Because the methods of the present invention are used to detect sepsis prior to a time that sepsis would be suspected using conventional techniques, the patient's disease status at early sepsis can only be confirmed retrospectively, when the manifestation of sepsis is more clinically obvious. The exact mechanism by which a patient becomes septic is not a critical aspect of the invention. The methods of the present invention can detect changes in the biomarker profile independent of the origin of the infectious process. Regardless of how sepsis arises, the methods of the present invention allow for determining the status of a patient having, or suspected of having, sepsis or SIRS, as classified by previously used criteria.

“Severe sepsis” refers to sepsis associated with organ dysfunction, hypoperfusion abnormalities, or sepsis-induced hypotension. Hypoperfusion abnormalities include, but are not limited to, lactic acidosis, oliguria, or an acute alteration in mental status. “Septic shock” refers to sepsis-induced hypotension that is not responsive to adequate intravenous fluid challenge and with manifestations of peripheral hypoperfusion.

A “biomarker” is virtually any biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, and a small molecule, that is present in the biological sample and that may be isolated from, or measured in, the biological sample. Furthermore, a biomarker can be the entire intact molecule, or it can be a portion thereof that may be partially functional or recognized, for example, by an antibody or other specific binding protein. A biomarker is considered to be informative if a measurable aspect of the biomarker is associated with a given state of the subject, such as a particular stage of sepsis. Such a measurable aspect may include, for example, the presence, absence, or particular concentration of the biomarker in the biological sample from the subject.

A “phenotypic change” is a detectable change in a parameter associated with a given state of the subject. For instance, a phenotypic change may include an increase or decrease of a biomarker in a bodily fluid, where the change is associated with sepsis or the onset of sepsis. A phenotypic change may further include a change in a detectable aspect of a given state of the patient that is not a change in a measurable aspect of a biomarker. For example, a change in phenotype may include a detectable change in body temperature, respiration rate, pulse, blood pressure, or other physiological parameter. Such changes can be determined via clinical observation and measurement using conventional techniques that are well-known to the skilled artisan. As used herein, “conventional techniques” are those techniques that classify an individual based on phenotypic changes without obtaining a biomarker profile according to the present invention.

“Isoform” is understood to mean proteins that have undergone alternative splicing or post-translational modifications. For example, a cell will generally transcribe and translate one gene into many related proteins. The differences in the proteins, called isoforms, are a result of alterations in the originally translated protein. There are over 800 known post-translational modifications which include, but are not limited to, protein cleavage fragments, phosphorylation, glycosylation, lipidation, cross-linking, protein folding, hydrogen bond interactions with other molecules, Van der Waals force interactions with other molecules, and covalent interactions with other molecules, etc.

Various aspects of the present invention are directed to the concept that Hp-MMP 9 complexes, produced by neutrophils in vitro and found in acute phase sera, have specific functional significance that differs from un-complexed forms of free Hp and MMP 9 alone. Further, serum concentrations of Hp-MMP 9 may serve as an independent indicator of clinically important events occurring during acute inflammation. The utility of Hp-MMP 9 complex ELISA in comparison to ELISA for un-complexed Hp or MMP 9 alone as an indicator of acute septic inflammatory disease in cattle is evaluated herein. Serum Hp-MMP 9 complexes are present in animals with acute, septic inflammation and may therefore provide a means to specifically identify systemic neutrophil activation in humans, cattle, and other mammals.

Thus, one aspect of the present invention provides a method of detecting whether a subject is suffering from an acute inflammatory condition or at risk of same by detecting the presence of a biomarker, or a particular concentration of a biomarker, in a sample from the subject. The sample may be a blood sample, such as the serum fraction. In one embodiment, the biomarker may include a protein, such as a serum protein. The biomarker may include the serum protein haptoglobin, or isoforms of the serum protein haptoglobin. In one embodiment, the biomarker may include a protein, such as a serum protein, in complex with an enzyme, such as a matrix metalloproteinase, such as matrix metalloproteinase 9 (an Hp-MMP 9 complex). The biomarker may include isoforms of the Hp-MMP 9 complex. In one embodiment, the method may include determining the concentration of the biomarker in samples collected from a subject. The method of this embodiment may also include comparing that concentration to a range of standard concentrations. In another embodiment, the method may include comparing the level of the biomarker in samples collected from the subject at different times. In yet another embodiment, the method may include comparing the biomarker with a reference biomarker from the same subject or a different subject.

While a specific embodiment is directed to Hp-MMP 9 as a biomarker, those of ordinary skill in the art will recognize that other tests using other markers may be developed using the guidance and techniques described herein. Exemplary biomarkers may include a protein, such as a serum protein, a nucleic acid or nucleotide, a carbohydrate, a lipid, a glycoprotein, a glycolipid, a protein fragment, a hormone, a steroid, a cytokine, a lymphokine, a chemokine, an immune modulator, a cell, a hematologic parameter, genomic expression from a cell in the sample, and combinations thereof. In one embodiment, the biomarker is an isoform of a serum protein, such as haptoglobin, transferrin, hemopexin, albumin, and combinations thereof. In one embodiment, the biomarker includes haptoglobin. In another embodiment, the biomarker may include a matrix metalloproteinase. The biomarker may include complexes of proteins as described above. In one particular embodiment, the biomarker may include a complex of haptoglobin and matrix metalloproteinase 9 (Hp-MMP 9 complex). In some embodiments, the biomarker is an isoform of a protein.

Examples of samples used for detecting biomarkers include whole blood, fractions of blood, such as the serum fraction, the plasma fraction, or the cellular fraction, such as white blood cells or red blood cells, other fluids (i.e., saliva, urine, cerebral spinal fluid, bile, extracellular fluid, cytosolic fluid, etc.), cells, tissues (such as skin, bone, muscle, hair, etc.), and combinations thereof.

The biomarker and levels of biomarker may be detected with a compound that selectively binds the biomarker. The selective compound can include a polyclonal antibody, a monoclonal antibody, an antibody fragment, a receptor, a phage display, a peptide, a protein fragment, a nucleic acid, a sequence of nucleic acids, a ribonucleic acid, a deoxyribonucleic acid, a small molecule, and combinations thereof. The selective compound may be coupled to or associated with a detection system to indicate the level of the detected biomarker. Exemplary detection systems may have elements that include, but are not limited to a test strip, chip, slide, microarray, titer plate, membrane, electrode, probe, bead, column, matrix, gel, and liquids. However, it will be recognized by those of skill in the art that detection may occur other than by binding the biomarker. For example, biomarker levels may alternatively be detected by analyzing a chemical parameter such as the size, charge, structure, and/or sequence of the biomarker.

The biomarker or levels of the biomarker may be detected using enzyme-linked immunosorbent assay (ELISA), gel electrophoresis, immunoprecipitation, radio-immunoassay, protein blotting, a test strip, chromatography, liquid chromatography, gas chromatography, binding assays, mass spectrometry, microarray, genomic microarray, polymerase chain reaction (PCR), Reverse transcription PCR (RT-PCR), real time RT-PCR, and combinations thereof. In one embodiment, the biomarker is detected with ELISA.

In one embodiment, a comparison of the level of the biomarker may be detected by comparing the level of the biomarker in at least two samples collected from the subject. The two samples can be collected at different times or from different sources. The level of the biomarker from the first sample is compared with the level of the biomarker in the second sample to determine the presence of increased Hp-MMP 9 in the subject when the amount in the second taken sample is greater than in the first taken sample.

In another embodiment, the level of the biomarker may be detected by comparing the level of the biomarker with the level of a reference biomarker. This could indicate the presence or absence of a condition, such as an acute inflammatory condition or risk of same.

In one embodiment, the reference biomarker is from another subject. Another subject in this context is understood to mean one or more other subjects and can represent the levels of reference biomarkers found in a population of subjects that are found to be indicative of either the presence or absence of an acute inflammatory condition or risk of same.

The invention also includes a kit for use in the methods described above for detecting the presence of Hp-MMP 9 in a subject. Exemplary uses for the kit include determining the presence of an inflammatory condition or risk of same in the tested subject. The kit includes devices and reagents configured to detect the presence of and levels of a biomarker (e.g., Hp-MMP 9) in a sample from the subject.

Another aspect of the invention provides a test for performing the method described above. In one embodiment, the test may be an ELISA.

One ELISA test embodiment is directed to a method for detecting multiple isoforms of a haptoglobin-matrix metalloproteinase 9 (Hp-MMP 9) complex in a biological sample. The method includes: (1) incubating a biological sample with a capture reagent immobilized on a solid support to bind multiple isoforms of Hp-MMP 9 to the capture reagent, wherein the capture reagent includes an antibody, such as a monoclonal antibody, that binds MMP9; and (2) detecting Hp-MMP 9 bound to the immobilized capture reagent by contacting the bound Hp-MMP 9 with a detectable antibody that binds to Hp.

Another ELISA test embodiment is directed to a process for identifying a patient with an acute inflammatory condition or at risk of an acute inflammatory condition by determining concentration of Hp-MMP 9 in a bodily fluid sample. The process includes (a) obtaining monoclonal antibodies specific for MMP9 and attaching the monoclonal antibodies to a solid support; (b) obtaining a sample of a bodily fluid from a patient wherein the sample is suspected of containing Hp-MMP 9 and/or immunogenic fragments of Hp-MMP 9; (c) adding the sample to the monoclonal antibodies of step (a) wherein the Hp-MMP 9 and/or immunogenic fragments of Hp-MMP 9 contained in the sample is captured by the monoclonal antibodies; (d) providing second antibodies specific for Hp; (e) labeling the second antibodies with a detector; (f) adding the second antibodies of step (e) to the Hp-MMP 9 and/or immunogenic fragments of Hp-MMP 9 captured in step (c) wherein the second antibodies of step (e) bind to the Hp-MMP 9 and/or immunogenic fragments of Hp-MMP 9 captured in step (c); (g) adding a reporter that reacts with the detector to form a reaction product; and (h) measuring the reaction product to determine concentration of Hp-MMP 9 in the sample; and (i) determining if the concentration of Hp-MMP 9 in the sample is elevated above a selected cut-off concentration indicative of concentration of Hp-MMP 9, wherein the elevated concentration identifies that the patient has an acute inflammatory condition or is at risk of an acute inflammatory condition.

Another ELISA test embodiment is directed to a method for detecting Hp-MMP 9 in plasma or serum. This test includes the steps of: (a) providing polyclonal or monoclonal antibodies against MMP9; (b) providing a microtiter plate coated with the antibodies; (c) adding the serum or plasma to the microtiter plate; (d) providing horseradish peroxidase-anti-Hp conjugates reactive with Hp to the microtiter plate; (e) providing hydrogen peroxide as a reactor to the microtiter plate; and (f) comparing the reaction which occurs as a result of steps (a) to (e) with a standard curve to determine the level of Hp-MMP 9 compared to a normal individual.

In one embodiment, the assay described herein is a multi-site immunoassay. In a first step of one embodiment of the assay, the biological sample is contacted and incubated with an immobilized capture (or coat) reagent or reagents, which may be an anti-MMP9 monoclonal antibody. These antibodies may be from any species, but the monoclonal antibody is often a murine monoclonal antibody. The monoclonal antibodies may be prepared by methods well known to those of ordinary skill in the art. For example, a mixture of affinity purified MMP 9 monomer and dimer produced by bovine neutrophils were used for immunization of BALB/C mice. Mice were immunized twice with 100 μg of purified MMP 9, over a 7 day interval. Four weeks later, mice were boosted with 100 μg of MMP 9 daily for 4 days and 4 days later, splenocytes were fused to the B cell myeloma SP2/0 and plated in 96 well plates. Hybridomas were selected in the presence of 100 μM hypoxanthine, 0.4 μM aminopterin, and 16 μM thymidine following established methods [as described in Kohler G, Milstein C (1975), Continuous cultures of fused cells secreting antibody of predefined specificity, Nature 256: 495-497, incorporated by reference herein in its entirety], well known to those of ordinary skill in the art. Clones that secreted antibodies with specificity for MMP 9 as tested by ELISA were further cloned by limiting dilution twice in order to ensure clonality [as described in Kohler G, Milstein C (1975), Continuous cultures of fused cells secreting antibody of predefined specificity, Nature 256:495-497, incorporated by reference herein]. Clone 10.1 was selected for positive reactivity and cellular amplification in a bioreactor [Celline, CL1000, Sartorius Stedim, North America, Inc. Bohemia N.Y. 11716], which was performed as follows.

The hybridoma cells were maintained in the cell compartment of the CL 1000 and harvested at approximately 7 day intervals. During harvest, cells and supernatant were collected from the cell compartment, a fraction of the harvest was reinoculated into the cell compartment with fresh medium. Nutrient medium was removed and replaced with fresh medium on day of harvest.

Methods

Murine hybridoma cell lines were thawed from frozen stocks and expanded in static culture (RPMI-1640, 10-15% FBS, 2× L-Glutamine, Pen-Strep). After demonstration of consistent cell doubling in static culture, cells were inoculated into the CL 1000 devices.

Cell Compartment Medium

RPMI-1640; 2× L-glutamine (5 mM), penicillin G (66 mg/L), streptomycin sulfate (144 mg/l). Basal medium was supplemented with 10% FBS (commercially available from Hyclone, Logan Utah). Additional supplementation of medium with a hybridoma growth supplement (0.1% Vitacyte, J. Brooks Irvine, Calif.)) was done to remain consistent with prior batch production runs of the same cell lines in traditional flasks.

Nutrient Medium

RPMI-1640; 2× L-glutamine (5 mM), penicillin G (66 mg/L) streptomycin sulfate (144 mg/l) with 0.8% FBS, 0.1% Vitacyte.

Inoculation

Cells were inoculated from static culture at Day 0 in a 20 ml volume into the cell compartment of the CL 1000 devices. Inoculation density was maintained above 3.0×10⁶ cells/ml. Cells were removed from frozen stock initiated cultures and resuspended in fresh cell compartment medium prior to inoculation. Nutrient medium (1000 ml) was supplied to the nutrient medium compartment and the devices placed into a 5% CO2, 37° C. humidified tissue culture incubator.

Harvest

At harvest, the total cell compartment volume was removed from the CL 1000 units by pipette. Cell numbers were determined by diluting and counting samples using a standard hemacytometer. Viable cells were discriminated from non-viable cells by trypan blue staining and phase contrast microscopy. Cell compartment contents were split back between 3-5 fold determined by cell numbers in the cell compartment at time of harvest. Fresh cell compartment medium (17-15 ml) was added to the cell fraction (3-5 ml) to achieve a 20 ml volume and the cell suspension returned to the cell compartment. The harvested cell containing supernatant fraction was kept sterile and stored at 4° C. until purification by affinity chromatography. Nutrient medium (1000 ml) was removed and replaced with fresh medium at the time the cell compartment was harvested. Devices were returned to incubator until next harvest. Devices were stacked atop of each other in the incubator.

Antibody purification

Culture supernatant was processed by eluting antibody from protein A affinity chromatography columns following manufacturers protocol. Eluted antibody fractions were collected, pooled and antibody quantified by spectrophotometer and ELISA. Sandwich ELISA was performed with polyclonal goat anti-mouse IgG or IgM capture antibody and polyclonal anti-mouse IgG or IgM antibody labeled with peroxidase. Color was developed with ABTS. Antibody purity was assessed by SDSPAGE and Coomassie blue staining.

Immobilization conventionally is accomplished by insolubilizing the capture reagents either before the assay procedure, as by adsorption to a water-insoluble matrix or surface (as described in U.S. Pat. No. 3,720,760) or non-covalent or covalent coupling [for example, using glutaraldehyde or carbodiimide cross-linking, with or without prior activation of the support with, e.g., nitric acid and a reducing agent as described in U.S. Pat. No. 3,645,852 or in Rotmans et al. J. Immunol. Methods 57:87-98 (1983)], or afterward, e.g., by immunoprecipitation.

The solid phase used for immobilization may be any inert support or carrier that is essentially water insoluble and useful in immunometric assays, including supports in the form of, e.g., surfaces, particles, porous matrices, etc. Examples of commonly used supports include small sheets, Sephadex®, polyvinyl chloride, plastic beads, and assay plates or test tubes manufactured from polyethylene, polypropylene, polystyrene, and the like including 96-well microtiter plates, as well as particulate materials such as filter paper, agarose, cross-linked dextran, and other polysaccharides. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are suitably employed for capture reagent immobilization. In one embodiment the immobilized capture reagents are coated on a microtiter plate, and in particular one exemplary solid phase used is a multi-well microtiter plate that can be used to analyze several samples at one time. One such ELISA plate is that sold as Dynatech Immobilon II (commercially available from Dynatech Laboratories, Chantily, Va.).

The solid phase is coated with the capture reagent(s), such as those described above, which may be linked by a non-covalent or covalent interaction or physical linkage as desired. Techniques for attachment include those described in U.S. Pat. No. 4,376,110 and the references cited therein. If covalent, the plate or other solid phase is incubated with a cross-linking agent together with the capture reagent under conditions well known in the art (such as for 1 hour at room temperature).

Commonly used cross-linking agents for attaching the pre-mixed capture reagents to the solid phase substrate include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates capable of forming cross-links in the presence of light.

If 96-well plates are utilized, they are generally coated with the mixture of capture reagents (typically diluted in a buffer such as TRIS buffered saline by incubation for at least about 12 hours, at temperatures of about 3° C. to 4° C., and at a pH of about 7.5.

The plates may be stacked and coated long in advance of the assay itself, and then the assay can be carried out simultaneously on several samples in a manual, semi-automatic, or automatic fashion, such as by using robotics.

The coated plates are then typically treated with a blocking agent that binds non-specifically to and saturates the binding sites to prevent unwanted binding of the free ligand to the excess sites on the wells of the plate. Examples of appropriate blocking agents for this purpose include, e.g., gelatin, bovine serum albumin (BSA), egg albumin, casein, and non-fat milk. In one particular embodiment, BSA, 20 mg/ml in TBS or SuperBlock Blocking Buffer are used as blocking agents. The blocking treatment typically takes place under conditions of ambient temperatures for about 1-4 hours.

After coating and blocking, the biological sample to be analyzed, appropriately diluted, is added to the immobilized phase. In certain embodiments, the dilution rate is about 2.5% (1:40) to 20% (1:5 dilution), such as about 10%, by volume. Buffers that may be used for dilution for this purpose include TBS+1 mg/mL BSA.

The amount of capture reagents employed is sufficiently large to give a good signal in comparison with the Hp-MMP 9 standards. For sufficient sensitivity, generally the amount of biological sample added is such that the immobilized capture reagents are in molar excess of the maximum molar concentration of free Hp-MMP 9 anticipated in the biological sample after appropriate dilution of the sample. This anticipated level depends mainly on any known correlation between the concentration levels of the free Hp-MMP 9 in the particular biological sample being analyzed with the clinical condition of the patient. In general, the concentration of all reagents should be tittered to obtain optimal results of the ELISA.

Thus, in these embodiments, while the concentration of the capture reagents will generally be determined by the concentration range of interest of the Hp-MMP 9 taking any necessary dilution of the biological sample into account, the final concentration of the capture reagents is determined empirically to maximize the sensitivity of the assay over the range of interest.

The conditions for incubation of sample and immobilized capture reagent are selected to maximize sensitivity of the assay and to minimize dissociation. The incubation is generally accomplished at fairly constant temperatures, ranging from about 3° C. to about 5° C., to obtain a less variable, lower coefficient of variant (CV) than at, e.g., room temperature. The time for incubation depends primarily on the temperature, being generally no greater than about 2 hours to avoid reduction of sensitivity of the assay. Generally, the incubation time is from about 2 hours at 3° C. to 5° C. to maximize binding of free Hp-MMP 9 to capture reagents.

At this stage, the pH of the incubation mixture will ordinarily be in the range of about 7.2-7.5. The pH of the incubation buffer is chosen to maintain a significant level of specific binding of the capture reagents to the Hp-MMP 9 being captured. Various buffers may be employed to achieve and maintain the desired pH during this step, including borate, phosphate, carbonate, Tris-HCl or Tris-phosphate, acetate, barbital, and the like. The particular buffer employed is not critical to the invention.

In the second step of the assay method herein, the biological sample is separated (by washing) from the immobilized capture reagents to remove uncaptured Hp-MMP 9. The solution used for washing is generally a buffer (“washing buffer') with a pH determined using the considerations and buffers described above for the incubation step, with a pH range of about 7.2-7.5. The washing may be done three or more times. The temperature of washing is generally from refrigerator to moderate temperatures, with a constant temperature maintained during the assay period, typically from about 22° C. to 25° C.

In the next step, any immobilized complexes are contacted with detectable antibodies, such as at a temperature of about 22° C. to 25° C., with the temperature and time for contacting the two being dependent primarily on the detection agent employed. For example, when horseradish peroxidase (HRP) and hydrogen peroxide are used as the means for detection, the contacting is generally carried out for 1 hour to 2 hours to amplify the signal to the maximum. The detectable antibody may be a polyclonal or monoclonal antibody. Also, the detectable antibody may be directly detectable, and may have a fluorimetric or colorimetric label.

Generally, a molar excess of an antibody with respect to the maximum concentration of captured Hp-MMP 9 expected (as described above) is added to the plate after it is washed. This antibody (which is directly or indirectly detectable) may be a polyclonal antibody, although any antibody can be employed. The affinity of the antibody is generally sufficiently high that small amounts of the free Hp-MMP 9 can be detected, but not so high that it causes non-specific background binding of the detectable antibodies.

In the last step of the assay method, the amount of Hp-MMP 9 that is now bound to the capture reagents is measured using a detection agent for the detectable antibody. Thus, the antibody added to the immobilized capture reagents will be either directly labeled, or detected indirectly by addition, after washing off of excess first unlabeled detectable antibody, of a molar excess of a second, labeled antibody directed against the first antibody.

The label used for either the first or second antibody may be any detectable functionality that does not interfere with the binding of free Hp-MMP 9 to the antibody. Examples of suitable labels are those numerous labels known for use in immunoassay, including moieties that may be detected directly, such as fluorochrome, chemiluminscent, chromogenic, and radioactive labels, as well as moieties, such as enzymes, that can be reacted or derivatized to be detected. Examples of such labels include horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, biotin/streptavidin, biotin/Streptavidin-β-galactosidase with MUG, spin labels, bacteriophage labels, stable free radicals, the radioisotopes ³²P, ¹⁴C, ¹²⁵I, ³H, and ¹³¹I, fluorophores such as rare earth cheats or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, and the like.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the antibodies with the above-described fluorescent, chemiluminescent, and enzyme labels. See, for example, U.S. Pat. No. 3,940,475 (fluorimetry) and U.S. Pat. No. 3,645,090 (enzymes); Hunter et al. Nature 144:945 (1962); David et al. Biochemistry 13:1014-1021 (1974); Pain et al. J. Immunol. Methods 40:219-230 (1981); and Nygren J. Histochem. and Cytochem. 30:407-412 (1982).

The conjugation of such label to the antibody is a standard manipulative procedure for one of ordinary skill in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Following the addition of last labeled antibody, the amount of bound antibody is determined by removing excess unbound labeled antibody through washing and then measuring the amount of the attached label using a detection method appropriate to the label, and correlating the measured amount with the amount of free Hp-MMP 9 in the biological sample. For example, in the case of enzymes, the amount of color developed and measured will be a direct measurement of the amount of Hp-MMP 9 present.

Kits

As a matter of convenience, the assay method of this invention can be provided in the form of a kit. Such a kit is a packaged combination including the basic elements of: (1) capture reagents comprised of polyclonal and monoclonal antibodies against human Hp-MMP 9 molecule; and (2) detection reagents comprised of detectable (labeled or unlabeled) antibodies that bind to Hp-MMP 9.

Generally, the kit further comprises a solid support for the capture reagents, which may be provided as a separate element or on which the capture reagents are already immobilized. Hence, the capture antibodies in the kit may be immobilized on a solid support, or they may be immobilized on such support that is included with the kit or provided separately from the kit. For example, the capture reagents may be coated on a microtiter plate. The detection reagent may be labeled antibodies detected directly or unlabeled antibodies that are detected by labeled antibodies directed against the unlabeled antibodies raised in a different species. Where the label is an enzyme, the kit will ordinarily include substrates and cofactors required by the enzyme, and where the label is a fluorophore, a dye precursor that provides the detectable chromophore. Where the detection reagent is unlabeled, the kit may further comprise a detection agent for the detectable antibodies, such as the labeled antibodies directed to the unlabeled antibodies, such as in a fluorimetric-detected format.

The kit also typically contains instructions for carrying out the assay, and/or Hp-MMP 9 as an antigen standard (e.g., purified Hp-MMP 9), as well as other additives such as stabilizers, washing and incubation buffers, and the like.

The components of the kit may be provided in predetermined ratios, with the relative amounts of the various reagents suitably varied to provide for concentrations in solution of the reagents that substantially maximize the sensitivity of the assay. Particularly, the reagents may be provided as dry powders, usually lyophilized, including excipients, which on dissolution will provide for a reagent solution having the appropriate concentration for combining with the sample to be tested.

The various aspects of the present invention will be described in greater detail with respect to the following non-limiting Example.

### EXAMPLES

### Example 1

This Example demonstrates that Hp-MMP 9 complexes have a specific diagnostic significance that differs from uncomplexed forms of free Hp and MMP9 alone. And,the present example describes an ELISA that can be used as a test for Hp-MMP 9 complexes, as well as ranges of Hp-MMP 9 complex concentration that can be used as an indicator of an acute inflammatory condition, or risk of same.

Materials and Methods

Animals

35 cattle were tested in this Example. The animals were those admitted to The Ohio State University VTH for evaluation of a variety of disorders (22 animals) or sampled from the Ohio State University Waterman Dairy (13 animals). Animals were either bled for routine clinico-pathologic work-up or as part of a herd infectious disease survey. Serum was harvested in routine fashion by centrifugation after clotting and frozen at −20° C. until analyzed. Animal age (3.5±1.7 yr; range 0.4-8 yr), breed (Holstein=20, Jersey=8, Hereford=2, Guernsey=1, Angus=3; Brown Swiss=1), sex (M=3, F=32) were recorded from patient and/or herd records (and are disclosed in Tables 1A, 1B, and 10, below). Cattle were grouped into (1) acute septic disease (15 cattle), (2) chronic disease (10 cattle) and (3) normal animals (10 cattle) based upon clinical signs, history and the results of clinicopathologic and pathologic evaluation in those animals who died or were euthanized. As these animals were examined by hospital veterinarians, they were classified as to their disease category prior to the analysis of the serum proteins by ELISA. The individual analyzing the samples by ELISA was not aware of the individual animal's identity or classification and was only given frozen serum samples with the animal's laboratory designation. The animals selected for inclusion were done so under the guidance of The Ohio State University VTH Hospital Executive Committee clinical investigation approval (Jan. 1, 2006 to Jan. 1, 2008) and an approval of laboratory studies obtained from The Ohio State University Institutional Animal Care and Use committee.

Purification of MMP 9 Molecular Species

Purification of bovine MMP 9 monomer, dimer and Hp-MMP 9 complexes was performed as described previously [in Bannikov G A, Mattoon J S, Abrahamsen E J, Premanandan C, Green-Church K B, Marsh A E, Lakritz J (2007), Biochemical and enzymatic characterization of purified covalent complexes of matrix metalloproteinase-9 and haptoglobin released by bovine granulocytes in vitro, Am. J. Vet. Res. 68:995-1004, incorporated by reference herein in its entirety]. In brief, neutrophils from fresh cow blood, were stimulated with PMA and conditioned media was subjected to reactive Red 120 Agarose (Sigma) chromatography, Gelatin Agarose affinity chromatography, and Ultragel AcA 34 gel-filtration. Fractions were analyzed by SDS PAGE, pooled, concentrated and stored at −20° C.

Monoclonal Antibody Production

A mixture of affinity purified MMP 9 monomer and dimer produced by bovine neutrophils were used for immunization of BALB/C mice. Mice were immunized twice with 100 μg of purified MMP 9, over a 7 day interval. Four weeks later, mice were boosted with 100 μg of MMP 9, sacrificed after 4 days, and splenocytes were fused to the B cell myeloma SP2/0 and plated in 96 well plates. Hybridomas were selected in the presence of 100 μM hypoxanthine, 0.4 μM aminopterin, and 16 μM thymidine following established methods [as described in Kohler G, Milstein C (1975), Continuous cultures of fused cells secreting antibody of predefined specificity, Nature 256:495-497, incorporated by reference herein in its entirety], well known to those of ordinary skill in the art. Clones that secreted antibodies with specificity for MMP 9 as tested by ELISA were further cloned by limiting dilution twice in order to ensure clonality [as described in Kohler G, Milstein C (1975), Continuous cultures of fused cells secreting antibody of predefined specificity, Nature 256:495-497]. Clone 10.1 was selected for positive reactivity and cellular amplification in a bioreactor [Celline, CL1000, Sartorius Stedim, North America, Inc. Bohemia N.Y. 11716] was performed as previously described. The reactivity of the hybridoma supernatant was tested using western immunoblot of purified monomer and dimer under reducing and non-reducing conditions and with enzymatically activated MMP 9 monomer and dimer. In addition, whole cell lysates from bovine neutrophils, and from peripheral blood leukocytes, were immunoblotted with the MAb. Immunoglobulin class (IgG1) of the MAb was determined using a commercial MAb isotyping kit [Pierce Rapid ELISA Mouse mAb isotyping kit, commercially available from Thermo Fisher Scientific, Rockford, Ill. 61105]. IgG from the hybridoma media was purified with Protein G agarose [commercially available from Thermo Fisher Scientific, Rockford Ill. 61105] and stored at concentration of 1 mg/ml at 4° C.

The anti-bovine MMP 9 monoclonal antibody (Clone 10.1) reacted positively in Western blot with bovine MMP 9 monomer and dimer whether reduced or not and after enzymatic activation. Clone 10.1 antibody also reacted positively in Western blot with recombinant MMP 9 of human origin.

Horse Radish Peroxidase (HRP) Conjugation of 10.1 Antibody

Approximately 1 mg of purified monoclonal antibody 10.1 was conjugated to HRP with the Roche HRP protein labeling labeling kit, in accordance with the manufacturers instructions [Peroxidase Labeling Kit, commercially available from Roche Diagnostics Corporation, Indianapolis, Ind. 46250]. The resulting antibody-HRP conjugate was purified by gel-filtration on Ultragel AcA 34 (Sigma, U8878) [commercially available from Sigma-Aldrich Co., St. Louis, Mo. 63178] and stored at 4° C. at a concentration 60 μg/ml.

ELISA Assays

Serum Hp ELISA—Serum Hp concentrations were determined using commercial Bovine haptoglobin ELISA test kits [Bovine haptoglobin 96-well ELISA, commercially available from Life Diagnostics, West Chester Pa. 19380], according to manufacturers instructions. Standard curves were prepared using purified bovine haptoglobin standard (2.5 μg/mL) included with the kit at a concentration range from 7.8-500 ng/mL. Serum samples were diluted according to the kit instructions (1:2,000 dilution) or used at lower dilution for samples containing low concentration of Hp and were run in duplicate. Controls included were normal bovine serum, 5% BSA in TBS, and blank wells.

Serum MMP 9 ELISA—The ELISA for bovine MMP 9 was developed in the laboratory, exploiting un-conjugated bovine MMP 9 MAb 10.1 as a capture antibody (100 μl, containing 1 μg per well) and HRP conjugated 10.1 antibody (0.3 μg/ml in TBS, 1 mg/ml BSA) for detection of bound MMP 9. These concentrations were chosen on basis of preliminary experiments. After capture antibody binding, the plates were washed 3×5 minutes with TBS and the wells blocked with bovine serum albumin [Bovine Serum Albumin, Fraction V, commercially available from Fisher Scientific, Thermo Fisher Scientific, Pittsburg Pa. 15275], at a concentration of 20 mg/ml in TBS or with Super Block Blocking Buffer [Pierce Super Block Blocking buffer, commercially available from Thermo Fisher Scientific, Rockford Ill. 61105]. After washing the blocked wells, and addition of serially diluted serum samples or known concentrations of purified bovine MMP 9 monomer (60 minutes at 22° C.), the plates were again washed 5×5 minutes with TBS and HRP conjugated MAb (10.1) added to each well at a final concentration of 1:200. The excess HRP-conjugate was removed by washing after which 100 μL of TMB added to each well for determination of HRP activity. The wells were incubated and colorimetric analysis followed over time using a microtiter plate reader (λ=630 nm). When color developed was maximal in the highest concentration standards, the reaction was stopped by addition of 1N hydrochloric acid. The color development was then determined using λ=450 nm. Quantitation of MMP 9 protein was determined using purified bovine MMP 9 protein as a calibration curve covering a range of concentrations from 39 to 2500 ng/ml. Samples registering lower than <39 ng/mL were considered to be zero.

Serum samples from sick and healthy animals were analyzed by serially diluting each sample with TBS containing 1 mg/ml BSA in duplicate. Once each set of samples was analyzed, and the absorbance value from the dilution which lay within the linear range of the standard curve was used to quantitate MMP 9 concentrations. For analysis sera were serially diluted with PBS, containing 1 mg/ml BSA and run in duplicate. Controls included were normal bovine serum, 5% BSA in TBS and blank wells.

Serum Hp-MMP 9 complex ELISA—Purified MAb 10.1 was used as a capture antibody as described above (100 μl, containing 1μg per well). However, the detection antibody chosen was an affinity purified HRP-conjugated rabbit anti-bovine haptoglobin [obtained from Immunology Consultants Laboratory, RHPT-10P, Newberg, Oreg. 97132] used at a final concentration of 0.033 μg/ml in TBS, 1 mg/ml BSA. These concentrations of both capture and detection antibodies were chosen on basis of preliminary experiments. Bovine serum albumin, 20 mg/ml in TBS or Super Block Blocking Buffer, were used as blocking agents. Purified bovine Hp-MMP 9 complexes were used to create standard curves using concentrations of 40-5000 ng/ml. Samples registering lower than 40 ng/mL were considered to be zero ng/mL. Once each set of samples were analyzed, the absorbance value from the dilution which lay within the linear range of the standard curve was used to quantitate Hp-MMP 9 concentrations. For analysis sera were serially diluted with PBS, containing 1 mg/ml BSA and run in duplicate. Controls included were normal bovine serum, 5% BSA in TBS and blank wells.

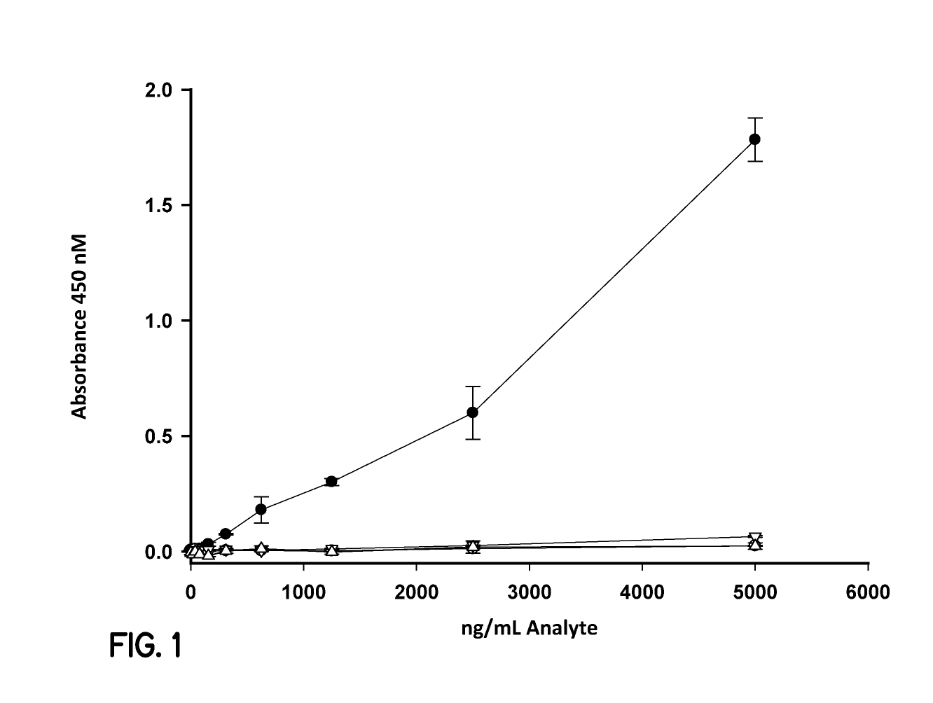

Specificity of serum Hp-MMP 9 ELISA—Affinity purified bovine Hp, MMP 9 monomer and Hp-MMP 9 complexes obtained as described above were used. In plates where the capture antibody was the anti-bovine MMP 9 monoclonal antibody (clone 10.1), increasing concentrations of Hp, MMP 9 and Hp-MMP 9 complexes (over the range of 4.8-5,000 ng/mL) were added to separate wells and allowed to bind. After washing, HRP conjugated secondary antibodies were added as follows: 1) To wells where Hp was added, anti-bovine Hp HRP conjugate or anti-bovine MMP 9 HRP conjugate was added. 2) To wells where affinity purified MMP 9 was added, anti-bovine Hp HRP conjugate or anti-MMP 9 monoclonal antibody HRP conjugate were added. 3) Finally, to wells where Hp-MMP 9 complexes were added, anti-bovine Hp HRP conjugate or anti-bovine MMP 9 HRP conjugate were added. After addition of TMB substrate, quantitation of antibody binding by determination of absorbance of each well as determined by the plate reader at 450 nm (FIG. 1).

Validation of ELISAs Results by Western Blot

To confirm the presence of Hp-MMP 9 complexes in individual serum samples detected by ELISA, an independent method was used. Gelatin bound fractions of selected sera were analyzed by Western bloting. Before SDS-PAGE analysis, 3 ml of each sera was adjusted to 0.5M NaCl and incubated with 300 μl of Gelatin Agarose previously equilibrated with 0.5M NaCl, 2 mM CaCl2, 20 mM Tris-HCl pH 7.5. After exhaustive washing, bound protein was eluted from the gelatin agarose with 3 ml of a buffer containing 20 mM Tris-HCl pH 7.5, 10 mM NaCl, 2 mM CaCl2, 0.015% Brij 35 and 10% DMSO. Eluates were diluted to 6 ml with the same buffer, without DMSO and concentrated to 300 μl in iCon concentrators [Pierce Concentrators, 9K MWCO, 7 mL, commercially available from Thermo Fisher Scientific. Rockford, Ill. 61105]. After dilution to 2 ml samples were again concentrated to 200 μl in iCon concentrators. Samples were diluted with 4× LDS sample buffer and electrophoresed through 4-20% Tris-Glycine gels [MuPAGE LDS sample buffer (4×), commercially available from Invitrogen Corp., Carlsbad, Calif. 92008; Novex 4-20% Tris-Glycine gel 1.0mm, 12 well, commercially available from Invitrogen Corp., Carlsbad, Calif. 92008]. Molecular mass markers were placed into one well to determine the approximate molecular mass of proteins eluted from gelatin affinity matrix [MagicMark XP Western protein standard (20-220 kDa), commercially available from Invitrogen Corp., Carlsbad, Calif. 92008]. Hp and MMP 9 bands were visualized by labeling with rabbit anti-bovine haptoglobin [Rabbit anti-bovine haptoglobin polyclonal antibody, commercially available from ICL, Inc., Newburg, Oreg. 97132] and goat anti-human MMP 9 [Goat anti-human MMP 9 affinity purified polyclonal AB, commercially available from R & D Systems Inc., Minneapolis, Minn. 55413]. HRP-conjugated anti-Rabbit and ant-Goat antibodies were used as secondary antibody followed by ECL visualization [20× Lumiglo reagent and 20× peroxide, commercially available from Cell Signaling Technology, Danvers, Mass. 01923].

Statistical Analysis

Animals were categorized by either (1) clinical diagnosis based upon the findings of clinical examination, laboratory, or other diagnostics, or (2) postmortem diagnosis based upon postmortem examination. Based on the results of those diagnoses, the animals were designated as acute septic disease; chronic or metabolic disease; or normal. Acute septic disease animals were desingated with the number “1,” chronic or metabolic disease animals were designated with the numeral “2,” and normal animals were designated with the numeral “3.” ELISA assays were performed by an individual without prior knowledge of the disease classification. Data from each animal, for each of the 3 ELISA assays were then placed into the spreadsheet by animal ID (Numbers 1-35).

Data from animals in each of the classifications, for each of the 3 ELISA assays were then described statistically (mean, standard deviation, minimum, maximum, median). Since the data were not normally distributed (zeros in normal and chronically ill animals for haptoglobin and haptoglobin-MMP 9 complex), non-parametric methods (Kruskal-Wallis ANOVA on ranks) were used to compare results for each of the 3 ELISA and each of the 3 groups. Further examination of the data included pairwise comparisons of the 3 ELISA assays using the Wilcoxin rank sum test. To account for multiple (n=3) pairwise comparisons, a Bonferoni adjustment was made and P-value<0.0167 (=0.05/3) was considered statistically significant.

Results

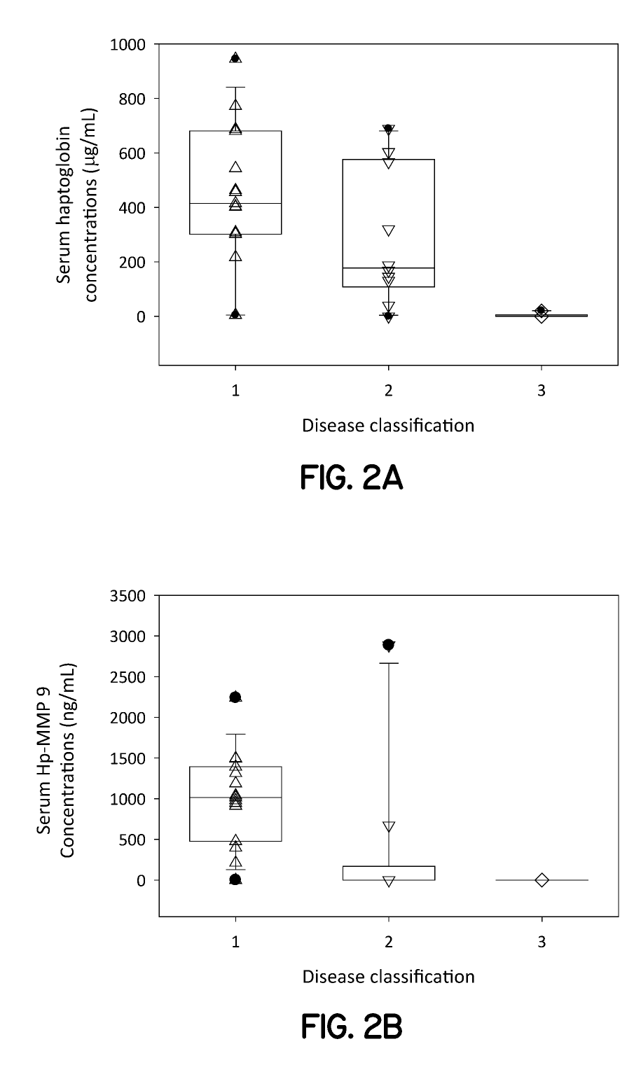

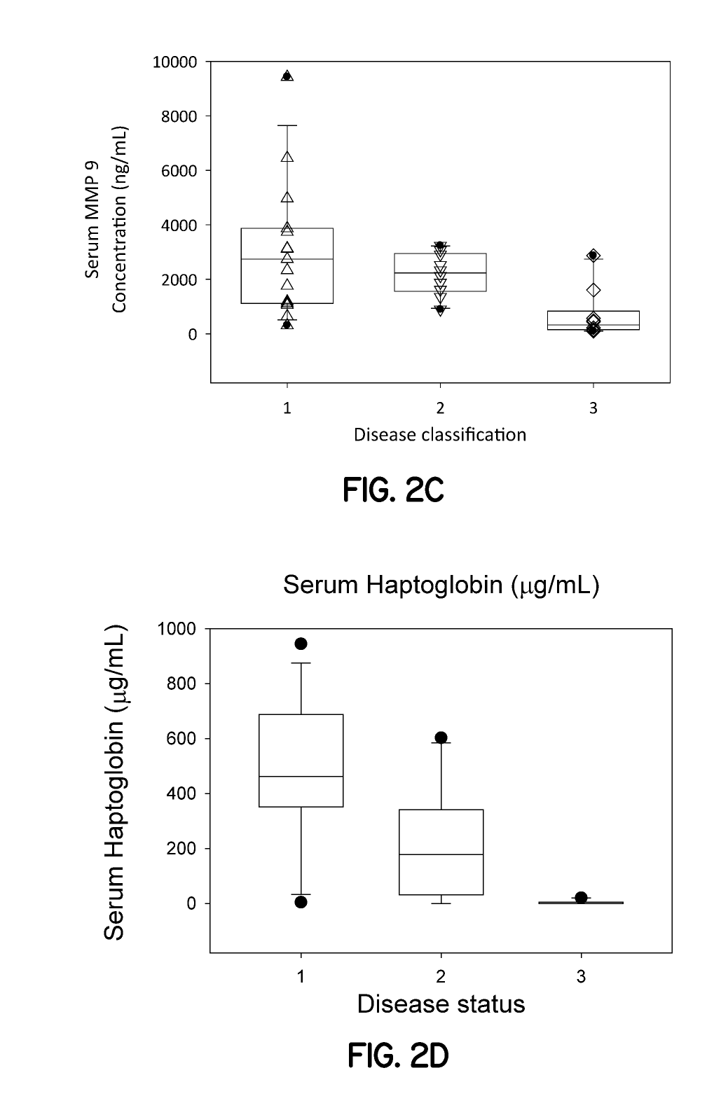

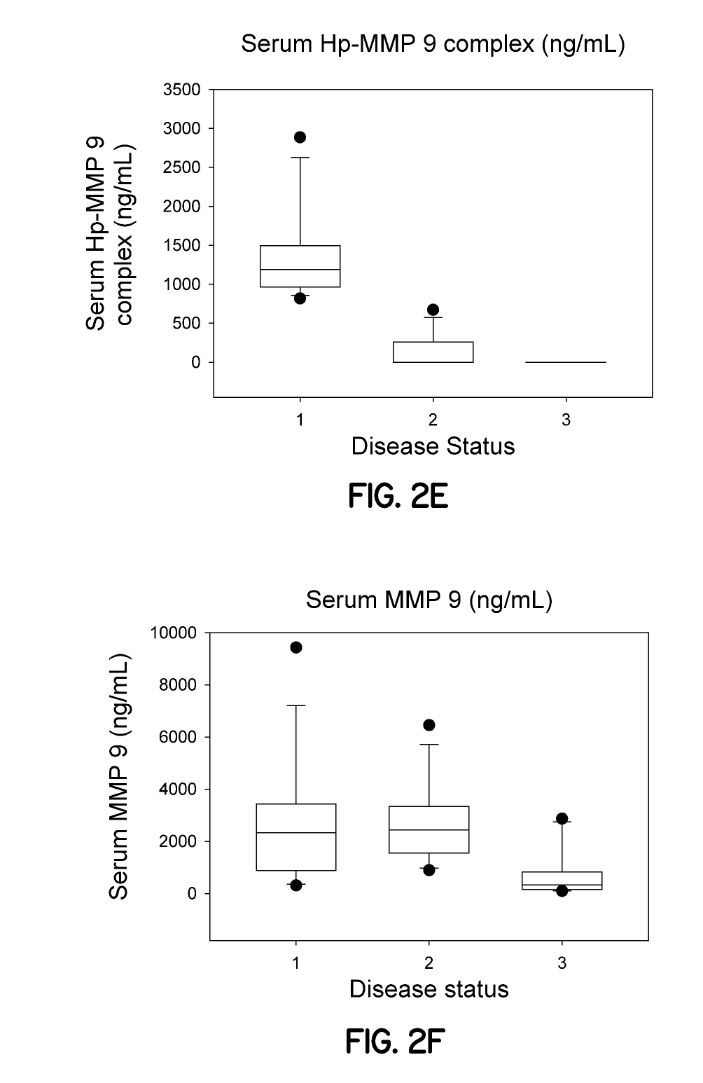

The anti-bovine MMP 9 monoclonal antibody (Clone 10.1) reacted positively in Western blot with bovine MMP 9 monomer and dimer whether reduced or not and after enzymatic activation. Clone 10.1 antibody also reacted positively in Western blot with recombinant MMP 9 of human origin. Analytical sensitivity of both MMP 9 ELISA and Hp-MMP 9 complex ELISA used in this study was 39 ng/ml. Analytical specificity of these assays is corroborated by the fact that irrelevant molecules (MMP 9 and Hp for Hp-MMP 9 complex ELISA and Hp-MMP 9 complex for MMP 9 ELISA) were not detectable up to concentrations 5000 ng/ml (FIG. 1). The history and the results of clinico-pathologic and pathologic evaluation of animals used in this study together with corresponding values of Hp, MMP 9 and Hp-MMP 9 complex in their sera are presented in Tables 1A, 1B, and 1C (below) and graphic representation of the distribution of the ELISA data for each animal in the three health status classifications are portrayed in FIGS. 2A, 2B, and 2C.

As will be discussed, in FIG. 2A, few normal animals had measureable serum Hp concentrations ( 8/10 were “0”), whereas serum Hp was high in acute septic and chronic inflammation/metabolic disease classifications. There was considerable overlap between Hp concentrations in serum of acute septic and chronic inflammation/metabolic disease classifications for this analyte. Median and 25th and 75th percentiles for serum haptoglobin, concentrations for acutely septic animals, animals with chronic or metabolic disease and for healthy animals are presented in Table 2. The serum haptoglobin concentrations in animals with acute inflammation (median 415 μg/mL; 25th-75th percentile 302, 680) and chronic inflammation/metabolic disease (median 178 μg/mL; 25th-75th percentile 131, 567) were not-significantly different from each other (p=0.1655). However, serum Hp concentrations in acute septic diseases and chronic inflammation/metabolic diseases were significantly greater than those observed in healthy cows (0 μg/mL; 25th-75th percentile 0, 0; p<0.0001, p=0.005, respectively).

Further, as will be discussed, in FIG. 2B, all normal and 8 of 10 disease classification 2 animals had no measureable serum Hp-MMP 9 concentrations. Animals grouped in disease classification 1 had measureable concentrations which were significantly greater than animals in Disease classification 2 and 3. Two disease classification 2 animals had elevated serum Hp-MMP 9 concentrations (FIG. 2B). One animal was diagnosed with thymic lymphoma and the other possibly had an acute disease that was not detected clinically. In acute septic animals, 14/15 animals possessed high serum concentrations of Hp-MMP 9 complexes (FIG. 2B). And as will be discussed, in FIG. 1C, animals in disease classification 1 and 2 had significantly greater serum MMP 9 than animals in disease classification 3. The serum concentrations of MMP 9 in disease classification 1 and 2 animals were not significantly different.

Statistical comparisons of each disease classification demonstrated that significant differences between normal and acutely ill animals were found for each of the analytes (Haptoglobin, MMP 9 and Hp-MMP 9 complexes). When comparing animals in disease category 2 (chronic disease) and 3 (normal cows), serum concentrations of haptoglobin and MMP 9 were statistically different. However, serum concentrations of Hp-MMP 9 complexes were not statistically significant in normal or chronically diseased animals.

All normal and 8 of 10 animals with chronic inflammation/metabolic disease had no measureable serum Hp-MMP 9 complexes. In acute septic animals, 14/15 animals possessed high serum concentrations of Hp-MMP 9 complexes (Table 1A, FIG. 2B)

Median and 25th and 75th percentiles for serum Hp-MMP 9 complexes in animals with acute septic disease (median 1014 ng/mL; 25th, 75th percentiles: 477, 1392 ng/mL) were statistically greater than those observed in animals with chronic inflammation/metabolic disease animals (median 0 ng/mL; 25th, 75th percentiles:0.0 ng/mL; p<0.0031) and in normal animals (median 0 ng/mL; 0.0 25th, 75th percentiles: 0.0 ng/mL; p<0.0001) (Table 2). The differences in serum concentrations of Hp-MMP 9 complexes between normal or chronically diseased animals were not statistically significant (p=0.1468).

The range of serum MMP 9 concentrations was much wider among animals with acute septic inflammation than among healthy animals and animals with chronic inflammation/metabolic disease (Table 2). There was, however, considerable overlap between the diseased animals (Table 10, FIG. 2C). Median and 25th and 75th percentiles for serum, free MMP 9 in animals with acute septic (median 2745 ng/mL; 25th, 75th percentiles: 1115, 3871 ng/mL), and with chronic/metabolic disease (median 2233 ng/mL; 25th, 75th percentiles: 1625, 2910 ng/mL) were significantly greater than those measured in healthy cattle (median 330 ng/mL; 25th, 75th percentiles: 153, 573 ng/mL; (p=0.0011 and P=0.0019, respectively) (Table 2). However, serum concentrations of MMP 9 in animals included in the acute disease and chronic/metabolic disease categories were not significantly different from each other (p=0.5417) (Table 2).

TABLE 1A
Disease classification 1—Acute disease in population of cattle
analyzed for serum haptoglobin, MMP 9 and Hp-MMP 9 complexes

| | | | | | | | HP- | | |
|---|---|---|---|---|---|---|---|---|---|
| Animal | Age | | | | Disease | HP | MMP9 | MMP9 | |
| # | (yr) | Breed | Sex | Diagnosis | Classification | (ug/ml) | (ng/ml) | (ng/ml) | Outcome |
| 1 | 2 | G | F | Peritonitis-Uterus/Gut | Acute | 544 | 1392 | 1115 | Euthanized |
| 2 | 3 | BS | F | Peritonitis-Gut | Acute | 308 | 212 | 3139 | Euthanized |
| 3 | 4 | Hol | F | Fibrinosuppurative | Acute | 945 | 2240 | 2745 | Euthanized |
| | | | | bronchopneumonia, | | | | | |
| | | | | endometritis | | | | | |
| 4 | 2 | J | F | Peritonitis-Gut | Acute | 415 | 947 | 2334 | Released |
| 5 | 5 | Hol | F | Peritonitis-Gut | Acute | 401 | 1315 | 1767 | Euthanized |
| 6 | 1 | A | M | Peritonitis-Gut | Acute | 687 | 1496 | 312 | Euthanized |
| 7 | 4 | Hol | F | Fibrinous bronchopneumonia | Acute | 772 | 1493 | 3871 | Euthanized |
| 8 | 4 | J | F | Coliform mastitis, metabolic | Acute | 217 | 0 | 1056 | Released |
| 9 | 6 | Hol | F | Peritonitis-Mammary vein | Acute | 403 | 399 | 4972 | Released |
| | | | | thrombophlebitis | | | | | |
| 10 | 3 | Hol | F | Peritonitis- ruptured Abscess | Acute | 463 | 913 | 646 | Euthanized |
| 11 | 3 | Hol | F | Peritonitis-Uterus | Acute | 456 | 1187 | 9433 | Euthanized |
| 12 | 2 | Hol | F | Peritonitis | Acute | 681 | 1014 | 3122 | Euthanized |
| 13 | 3 | Hol | F | Toxic mastitis | Acute | 5 | 477 | 6456 | Euthanized |
| 14 | 4 | Her | F | Septic caval | Acute on | 302 | 1039 | 1181 | Euthanized |
| | | | | thrombophlebitis/Hepatic | chronic | | | | |
| | | | | abscess/Fibrinosuppurative | | | | | |
| | | | | bronchopneumonia | | | | | |
| 15 | 4 | Hol | F | Endocarditis/Chronic | Acute on | 4 | 979 | 3742 | Euthanized |
| | | | | bronchopneumonia, | Chronic | | | | |

TABLE 1B
Disease classification 2—Chronic diseases in population of cattle
analyzed for serum haptoglobin, MMP 9 and Hp-MMP 9 complexes.

| | | | | | | | HP- | | |
|---|---|---|---|---|---|---|---|---|---|
| Animal | Age | | | | Disease | HP | MMP9 | MMP9 | |
| # | (yr) | Breed | Sex | Diagnosis | Classification | (ug/ml) | (ng/ml) | (ng/ml) | Outcome |
| 16 | 4 | Hol | F | Peritonitis-Surgical | Chronic | 603 | 0 | 2129 | Euthanized |
| 17 | 3 | Her | M | Thymic lymphoma | Chronic | 40 | 673 | 2336 | Euthanized |
| 18 | 4 | Hol | F | Chronic suppurative | Chronic | 0 | 0 | 1881 | Released |
| | | | | bronchopneumonia | | | | | |
| 19 | 4 | Hol | F | Metabolic, Surgical | Chronic | 689 | 2885 | 2910 | Released |
| | | | | inflammation | | | | | |
| 20 | 4 | Hol | M | Abomasal | Chronic | 131 | 0 | 1358 | Euthanized |
| | | | | lymphoma/Perforation/ | | | | | |
| | | | | Omental bursitis | | | | | |
| 21 | 0.4 | A | F | Chronic pneumonia | Chronic | 146 | 0 | 1625 | Euthanized |
| 22 | 0.7 | A | F | Chronic pneumonia | Chronic | 168 | 0 | 3240 | Released |
| 23 | 4 | Hol | F | Mastitis | Chronic | 567 | 0 | 895 | Released |
| 24 | 4 | Hol | F | Metritis | Chronic | 320 | 0 | 2531 | Released |
| 25 | 8 | J | F | Metritis/Retained Placenta | Chronic | 187 | 0 | 3078 | Released |

TABLE 1C
Disease classification 3—Normal animal population of dairy cattle
analyzed for serum haptoglobin, MMP 9 and Hp-MMP 9 complexes.

| | | | | | | | HP- | | |
|---|---|---|---|---|---|---|---|---|---|
| Animal | Age | | | | Disease | HP | MMP9 | MMP9 | |
| # | (yr) | Breed | Sex | Diagnosis | Classification | (ug/ml) | (ng/ml) | (ng/ml) | Outcome |
| 26 | 4 | Hol | F | Healthy | Normal | 0 | 0 | 153 | Released |
| 27 | 3 | Hol | F | Healthy | Normal | 20 | 0 | 440 | Released |
| 28 | 3 | Hol | F | Healthy | Normal | 0 | 0 | 100 | Released |
| 29 | 3 | Hol | F | Healthy | Normal | 0 | 0 | 480 | Released |
| 30 | 3 | Hol | F | Healthy | Normal | 0 | 0 | 143 | Released |
| 31 | 7 | J | F | Healthy | Normal | 0 | 0 | 220 | Released |
| 32 | 6 | J | F | Healthy | Normal | 0 | 0 | 2870 | Released |
| 33 | 5 | J | F | Healthy | Normal | 20 | 0 | 573 | Released |
| 34 | 6 | J | F | Healthy | Normal | 0 | 0 | 153 | Released |
| 35 | 4 | J | F | Healthy | Normal | 0 | 0 | 1606 | Released |

TABLE 2
Median and the 25^{th} and 75^{th} percentiles for serum haptoglobin, Hp-MMP
9 complex and MMP 9 in 35 animals classified as acute septic disease (1),
chronic inflammation/metabolic disease (2), and healthy cattle (3).

| | Haptoglobin | | Hp-MMP 9 | | MMP 9 | |
|---|---|---|---|---|---|---|
| | μg/mL | | ng/mL | | ng/mL | |
| Disease | | 25^{th}-75^{th} | | 25^{th}-75^{th} | | 25^{th}-75^{th} |
| Classification | Median | Percentile | Median | Percentile | Median | Percentile |
| 1 | 415 | 302, 680 | 1014 | 477, 1392 | 2745 | 1115, 3871 |
| Acute septic | | | | | | |
| 2 | 178 | 131, 567 | 0^{a} | 0, 0 | 2233 | 1625, 2910 |
| Chronic, metabolic | | | | | | |
| 3 | 0^{a} | 0, 0 | 0^{a} | 0, 0 | 330^{a} | 153, 573 |
| Normal | | | | | | |

Data are presented as the median value and 25^{th} and 75^{th} percentile values. Serum concentrations of Hp, Hp-MMP 9 and MMP 9 were compared between disease classification groups using the Kruskal-Wallis One way ANOVA on ranks with pair-wise comparisons using Wilcoxon rank-sum test with Bonferroni adjustment.
^{a}concentrations with a different superscript (e.g., one with and one without a superscript) within a column are statistically significantly different from each other at P < 0.003 level.

In comparisons between acute septic disease (disease category 1) and chronic disease (disease category 2) haptoglobin and haptoglobin-MMP 9 complex concentrations differed significantly. There was no difference in the MMP 9 concentrations between cattle in disease classification 1 and 2.

Discussion

This study compares the diagnostic utility of serum concentrations of matrix metalloproteinase 9 (MMP 9), haptoglobin (Hp) and haptoglobin-matrix metalloproteinase 9 (Hp-MMP 9) complexes in cattle with acute septic disease and chronic inflammatory, or metabolic disease in comparison to normal animals. This analysis was prompted by previous findings that Hp-MMP 9 in conditioned media from phorbol ester stimulated bovine neutrophils in vitro and the observation of the presence of Hp-MMP 9 in serum of septic cattle but not in serum chronically inflamed or healthy cows [Bannikov G A, Mattoon J S, Abrahamsen E J, Premanandan C, Green-Church K B, Marsh A E, Lakritz J (2007), Biochemical and enzymatic characterization of purified covalent complexes of matrix metalloproteinase-9 and haptoglobin released by bovine granulocytes in vitro, Am. J. Vet. Res. 68:995-1004]. The design of this study also included measurement of serum concentrations of two individual components of Hp-MMP 9 complex: Hp, which is a major acute phase protein in cattle and MMP 9 which concentrates in serum of cattle undergoing inflammation.

In order to conduct the present study, a monoclonal antibody to bovine MMP 9 and an ELISA assay that specifically recognizes either MMP 9 or Hp-MMP 9 complexes was developed. As determined experimentally, the Hp-MMP 9 ELISA does not recognize free serum MMP 9 or free serum Hp as individual molecules (FIG. 1). Furthermore, that the MMP 9 ELISA does not recognize Hp-MMP 9 complexes in serum (FIG. 1) was also confirmed. Finally, serum Hp-MMP 9 complexes in cattle with acute septic disease do not distort data for Hp-ELISA, because the serum concentrations of Hp-MMP 9 complexes are present in concentrations that are 3 orders of magnitude lower than the concentrations of Hp.

Studies have shown that MMP 9 is stored pre-formed in granules in peripheral blood neutrophils [Bannikov G A, Mattoon J S, Abrahamsen E J, Premanandan C, Green-Church K B, Marsh A E, Lakritz J (2007), Biochemical and enzymatic characterization of purified covalent complexes of matrix metalloproteinase-9 and haptoglobin released by bovine granulocytes in vitro, Am. J. Vet. Res. 68:995-1004; Klimiuk P A, Sierakowski S, Latosiewicz R, Cylwik B, Skowronski J, Chwiecko J (2002), Serum matrix metalloproteinases and tissue inhibitors of metalloproteinases in different histological variants of rheumatoid synovitis, Rheumatology. (Oxford) 41:78-87; Ohtsuka H, Kudo K, Mori K, Nagai F, Hatsugaya A, Tajima M, Tamura K, Hoshi F, Koiwa M, Kawamura S (2001), Acute phase response in naturally occurring coliform mastitis, J. Vet. Med. Sci. 63: 675-678]. In the present Example, MMP 9 was clearly elevated in acutely or chronically ill animals in comparison to clinically normal cows (Tables 1A-C, FIGS. 2A-C). On the other hand, the data demonstrated that serum concentrations of MMP 9 in chronic inflammatory/metabolic disease and acute septic disease of cattle are nearly identical, suggesting that serum MMP 9 is not suitable for differentiation of acute and chronic inflammatory or metabolic diseases. At this point, the use of serum MMP 9 concentrations as an acute phase protein cannot be recommended, since detection of this protein could lead to a false-positive diagnosis due to substantial variation of serum MMP 9 concentrations even in healthy cattle. The lack of clear-cut boundaries between concentrations of MMP 9 in all three groups of animals studied may be related to dual roles of MMP 9 in inflammation favoring either pro-inflammatory or anti-inflammatory activity [as has been suggested in Klimiuk P A, Sierakowski S, Latosiewicz R, Cylwik B, Skowronski J, Chwiecko J (2002), Serum matrix metalloproteinases and tissue inhibitors of metalloproteinases in different histological variants of rheumatoid synovitis, Rheumatology.(Oxford) 41:78-87; Larsen K, Macleod D, Nihlberg K, Gurcan E, Bjermer L, Marko-Varga G, Westergren-Thorsson G (2006), Specific haptoglobin expression in bronchoalveolar lavage during differentiation of circulating fibroblast progenitor cells in mild asthma, J. Proteome. Res. 5:1479-1483; Ohtsuka H, Kudo K, Mori K, Nagai F, Hatsugaya A, Tajima M, Tamura K, Hoshi F, Koiwa M, Kawamura S (2001), Acute phase response in naturally occurring coliform mastitis, J. Vet. Med. Sci. 63: 675-678].

Although Hp is recognized as an indicator of acute inflammation in cattle, moderate increases of serum Hp have been described for cows with hepatic lipidosis, despite having no clinically apparent signs of inflammation [Godson D L, Campos M, Attah-Poku S K, Redmond M J, Cordeiro D M, Sethi M S, Harland R J, Babiuk L A (1996), Serum haptoglobin as an indicator of the acute phase response in bovine respiratory disease, Vet. Immunol. Immunopathol. 51:277-292; Katoh N, Miyamoto T, Nakagawa H, Watanabe A (1999), Detection of annexin I and IV and haptoglobin in bronchoalveolar lavage fluid from calves experimentally inoculated with Pasteurella haemolytica, Am. J. Vet. Res. 60:1390-1395; Morimatsu M, Syuto B, Shimada N, Fujinaga T, Yamamoto S, Saito M, Naiki M (1991), Isolation and characterization of bovine haptoglobin from acute phase sera, J. Biol. Chem. 266:11833-11837; Peake N J, Foster H E, Khawaja K, Cawston T E, Rowan A D (2006), Assessment of the clinical significance of gelatinase activity in patients with juvenile idiopathic arthritis using quantitative protein substrate zymography, Ann. Rheum. Dis. 65:501-507]. Further, substantial variation in Hp concentrations in milk has been observed in cows with chronic, sub-clinical mastitis [Gronlund U, Hallen S C, Persson 489 WK (2005), Haptoglobin and serum amyloid A in milk from dairy cows with chronic sub-clinical mastitis, Vet. Res. 36:191-198]. Previous studies demonstrate that Hp concentration in sera of healthy cows is negligible (approximately 20 ng/mL or lower) but can increase in acute inflammation to values as high as 950 μg/mL.

On the other hand, a majority of the cows with chronic inflammation/metabolic diseases in the study also have considerable concentration of Hp in their sera. Because of the lack of statistical difference between Hp concentrations in chronic inflammatory/metabolic disorders and acute septic disease, it is believed that serum Hp concentrations, by themselves, are a poor discriminator between acute and chronic inflammation in cattle.

Data from the current study demonstrated a significant diagnostic advantage of the Hp-MMP 9 ELISA over the Hp and MMP 9 ELISA assays. There are significant differences in serum Hp-MMP 9 concentrations observed in cattle with acute septic disease compared to those animals with chronic inflammatory/metabolic disease or healthy animals. The data suggest that the Hp-MMP 9 assay is specific for acute, septic diseases. Of the 15 animals identified clinically as having acute inflammation, 14 animals had high serum concentrations of Hp-MMP 9. Only 1 of 15 animals had serum concentrations of Hp-MMP 9 complexes<39 ng/mL (interpreted as 0). This cow (#8; Table 1A), had recently calved and developed Klebsiella spp. mastitis. Serum concentrations of Hp and MMP 9 were within the ranges of those observed in the serum of both disease category 2 and 3 animals. However, this animal had been treated with Predef 2×™ (isoflupredone acetate) for 3 days. The lack of serum Hp-MMP 9 complexes may be related to prior corticosteroid treatment since corticosteroids have profound effects on inflammation in vitro and in vivo [Cohn, L. A. (2003), The influence of corticosteroids on host defense mechanisms, J. Vet. Intern. Med. 5:95-104].

The specificity of Hp-MMP 9 complex ELISA for acute septic condition is further corroborated by its presence in only 2 of 12 chronically ill animals. One of these two cases (Table 1 B, #16) was diagnosed with thymic lymphoma, without any pathologically described necrosis or bacterial infection. The presence of the Hp-MMP 9 complexes in cattle with neoplasia should be investigated. A second Hp-MMP 9 positive animal was placed into disease classification 2 based upon clinical findings (Table 1 B, #19). Although it did possess serum Hp-MMP 9 complexes, the lack of diagnostic testing performed on this particular animal prior to discharge, precludes an explanation of the presence of these serum Hp-MMP 9 complexes.

Another favorable feature of the Hp-MMP 9 ELISA was the narrow range of concentrations in the sera of acutely septic animals: median concentrations of Hp-MMP 9 in animals classified clinical as acute septic were 1,014 ng/mL (with 25th, 75th percentiles ranging from 586-1373 ng/mL). Median values for the chronic inflammation/metabolic disease cases were 0 ng/mL. The relatively narrow range of serum Hp-MMP 9 complex concentrations in acutely septic animals enhances its diagnostic utility.

The biological rationale for the specificity of an ELISA based assay for serum Hp-MMP 9 complexes likely consists of the uniqueness of its cellular origin (at present, the only known source is degranulating neutrophils) [Bannikov G A, Mattoon J S, Abrahamsen E J, Premanandan C, Green-Church K B, Marsh A E, Lakritz J (2007), Biochemical and enzymatic characterization of purified covalent complexes of matrix metalloproteinase-9 and haptoglobin released by bovine granulocytes in vitro, Am. J. Vet. Res. 68:995-1004]. In contrast, free serum Hp and free MMP 9 are produced and secreted by a number of cellular sources in response to a variety of challenges [D'Armiento J, Dalal S S, Chada K (1997), Tissue, temporal and inducible expression pattern of haptoglobin in mice, Gene 195:19-27; Sharpe-Timms K L, Zimmer R L, Ricke E A, Piva M, Horowitz G M (2002), Endometriotic haptoglobin binds to peritoneal macrophages and alters their function in women with endometriosis, Fertil. Steril. 78:810-819]. This is supported by the data showing 8 of 10 animals with chronic inflammation/metabolic disease, free Hp and free MMP 9 were elevated without the presence of measureable quantities of Hp-MMP 9 complexes.

In conclusion, when compared to free Hp or MMP 9, serum concentrations of Hp-MMP 9 appear to be a more reliable indicator of acute septic inflammation in cattle. Thus, application of the Hp-MMP 9 ELISA may be beneficial for the diagnosis of early events in acute septic conditions in the bovine.

### Example 2

The study in this Example demonstrates that, in vivo, LPS induced hematologic and biochemical alterations are associated with the presence of serum Hp-MMP 9 complexes, and so the complexes can serve as biomarkers of various conditions in cattle, such as toxemia.

Materials and methods

9 male calves approximately 6 weeks of age were obtained from the OSU Waterman Dairy. A single dose of bacterial LPS, specifically E. coli O111:B4 endotoxin was administered to each of the 9 calves intravenously at a ratio of 2.5 ug/Kg body weight. Serum samples were then obtained from each calf sequentially over 96 hours post-endotoxin administration by standard techniques that are known to those of ordinary skill in the relevant art (e.g., serum was harvested in routine fashion by centrifugation after clotting and frozen at −20° C. until analyzed).

The ELISA test performed on the serum samples is as described above in Example 1, though those of ordinary skill in the art will recognize that parameters can be modified (and validated), such as is described below in Example 6.

Results

Intravenous LPS infusion induces changes in serum cortisol, white blood cell activation and Hp-MMP 9.

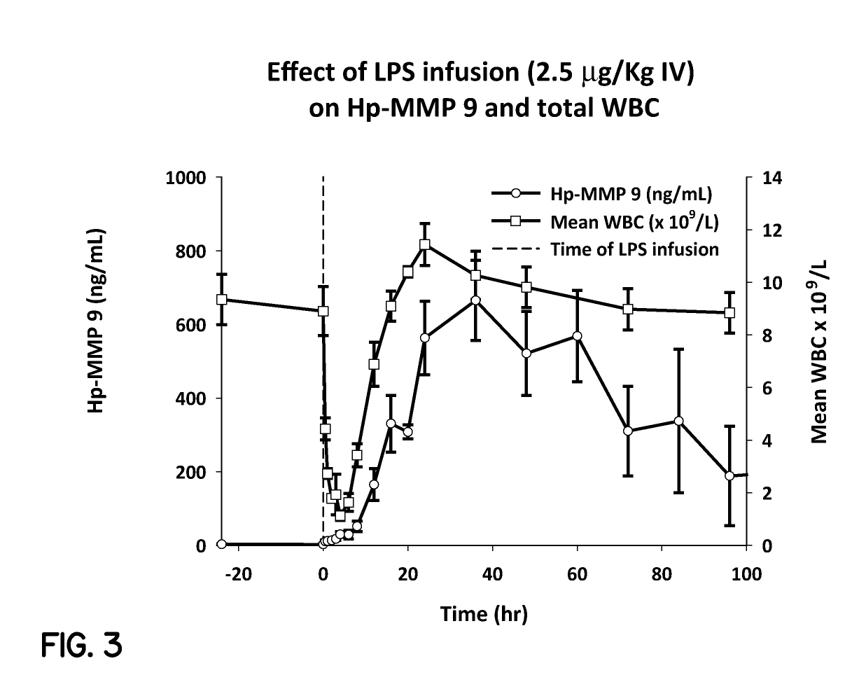

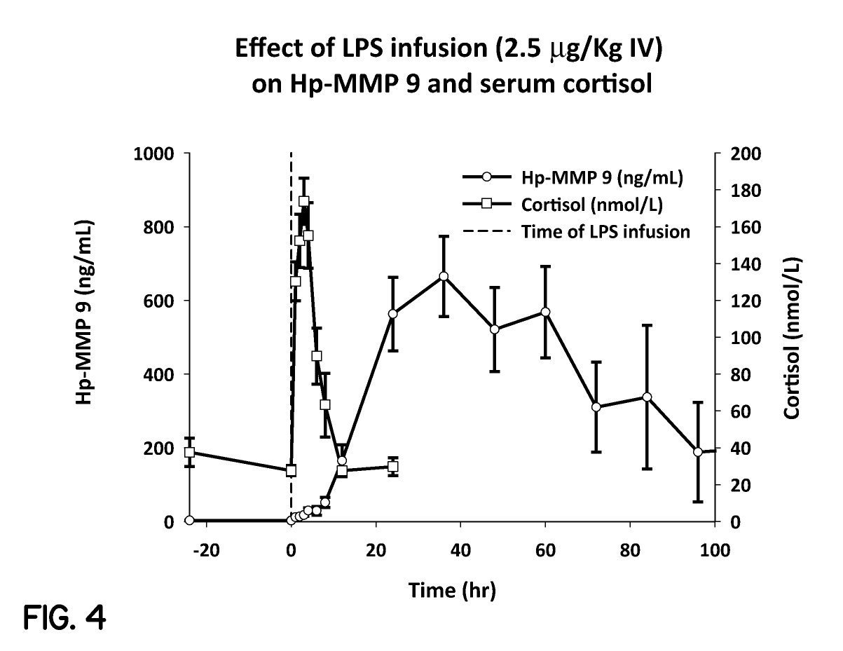

Intravenous LPS infusion was associated with several hematologic and biochemical findings of importance. Referring to FIG. 3, LPS infusion caused a rapid and statistically significant drop in peripheral blood white blood cell counts (P<0.01 compared to t=0; Mixed procedure, SAS). This was due to margination of neutrophils to the endothelial lining of the blood vessels, and rapid decline of lymphocyte numbers within the blood by 0.5 hours (P<0.001 compared to t=0; Mixed procedure, SAS). Further, referring now to FIG. 4, LPS infusion was associated with rapid elevation in serum cortisol <1 ug/mL to >4.7 ug/mL by 1 hour, peaking in these 9 calves to 6.3 ug/dL by 3 hours post-LPS infusion (P<0.001 compared to t=0; Mixed procedure, SAS). It is believed that the rapid increase in cortisol is likely responsible for the dramatic decline in lymphocyte number. Peripheral blood leukocyte counts remained low for approximately 4 hours post-LPS infusion, whereby cell counts gradually increased over the next 20 hours to return to normal.

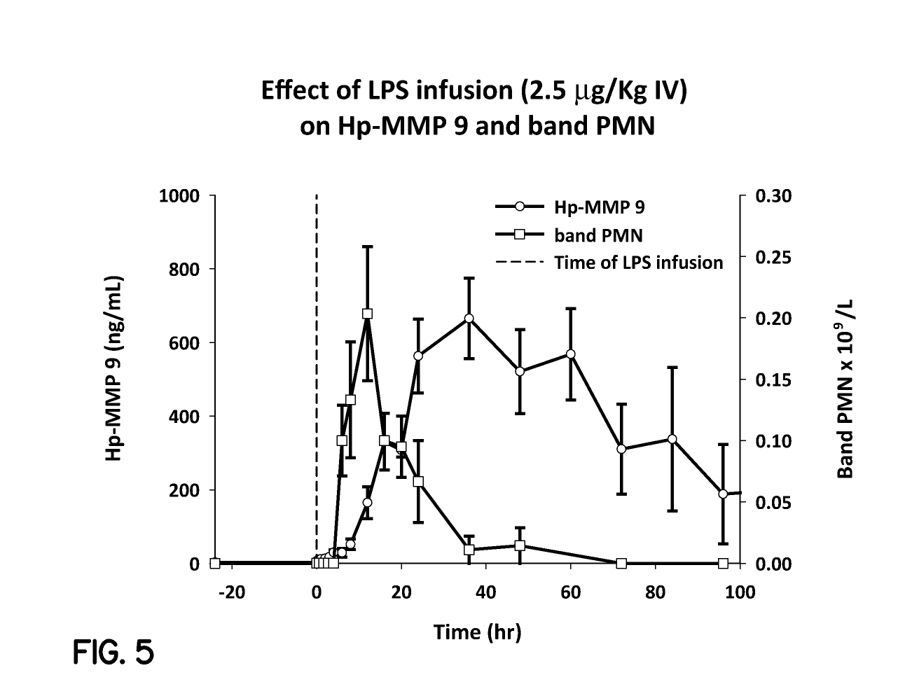

Referring now to FIG. 5, immature, band PMN increased from 0/uL to a mean of 600/uL approximately 8 hours pos-LPS infusion (P<0.001 compared to t=0; Mixed procedure, SAS). At approximately 6 hours post-LPS infusion, in association with the decline of serum cortisol (FIG. 4) and start of WBC rebound (FIG. 3) in peripheral blood, serum Hp-MMP 9 complexes were detectable, and increased to a peak at 36 hours post-LPS (FIGS. 3-5; P<0.01 compared to t=0; Mixed procedure, SAS). From 36-96 hours post LPS infusion, concentrations of Hp-MMP9 gradually declined within the serum.

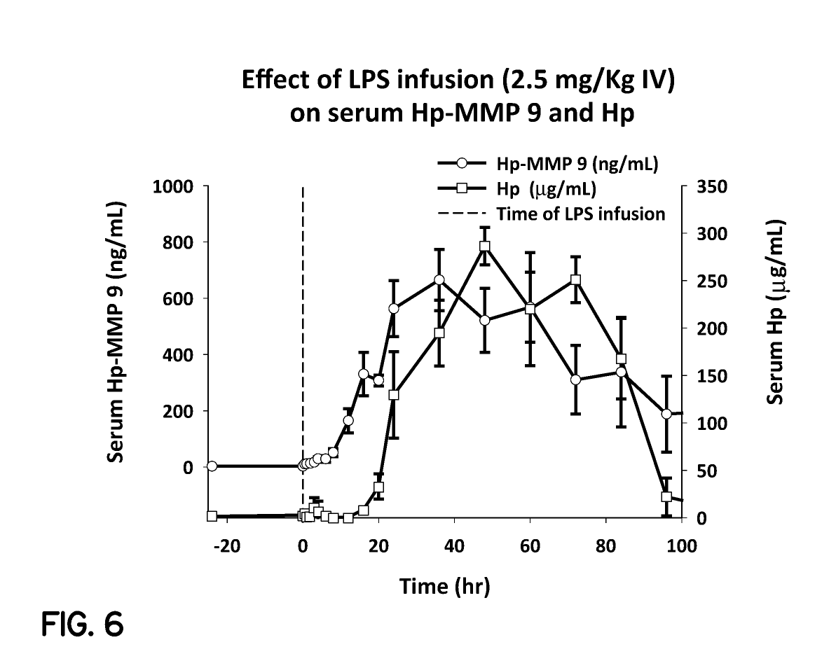

Similarly, and referring now to FIG. 6, when serum Hp-MMP 9 was detected (based upon the ELISA specific for Hp-MMP 9), serum total haptoglobin (Hp) remained below cut-off point, only exceeding this threshold at approximately 20 hours. The concentration versus time course of serum total haptoglobin was very similar in shape to that of serum Hp-MMP 9, albeit delayed by 6-10 hours after Hp-MMP 9 appearance (FIG. 6). Furthermore, the magnitude of serum total haptoglobin achieved was approximately half that observed in prior studies of LPS induced serum Hp. These LPS induced Hp-MMP 9 complexes and serum Hp were observed to return to normal within 96 hours, rather than 8 days as previously reported. Without being bound to any theory, it is believed that this may be due to initial release of neutrophil Hp-MMP 9 complexes, which would be detected when using the serum total haptoglobin ELISA.

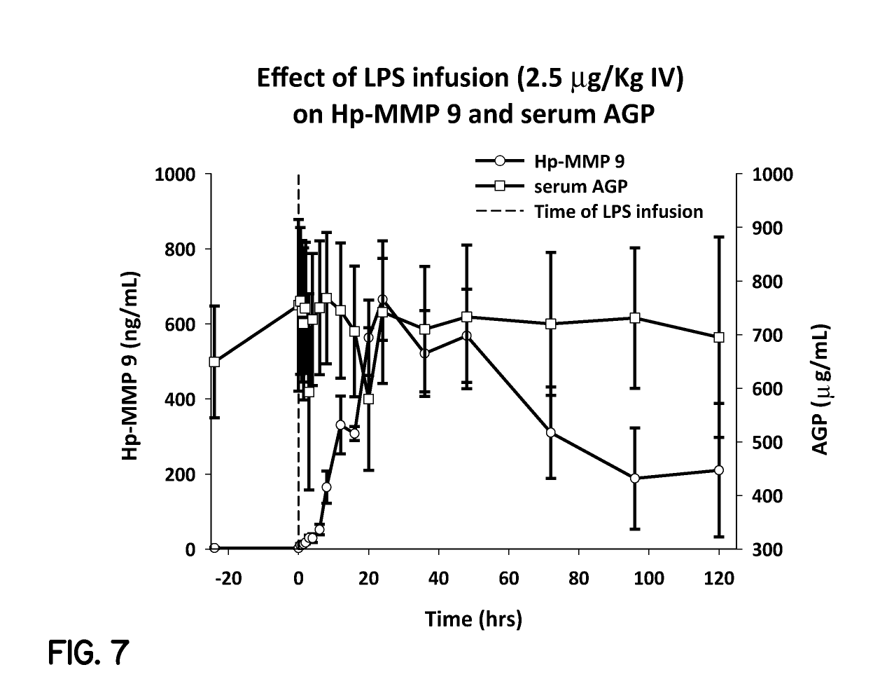

In contrast, and referring now to FIG. 7, another serum acute phase protein (alpha 1 acid glycoprotein—“AGP”) did not significantly change over the course of the study described in this Example. Like the serum Hp assay, this single radial immunodiffusion (SRID) format immunoassay measures total AGP. Like previously reported studies, serum AGP does not appear to provide similar information to that associated with serum Hp-MMP 9 complexes. These data suggest that the release of serum Hp-MMP 9 is an earlier marker of neutrophil activation, occurring in association with the rapid decline and increase in peripheral blood leukocytes associated with intravenous LPS.

### Example 3

This Example shows that experimental Mycoplasma bovis lung infection is associated with serum Hp-MMP 9 complexes.

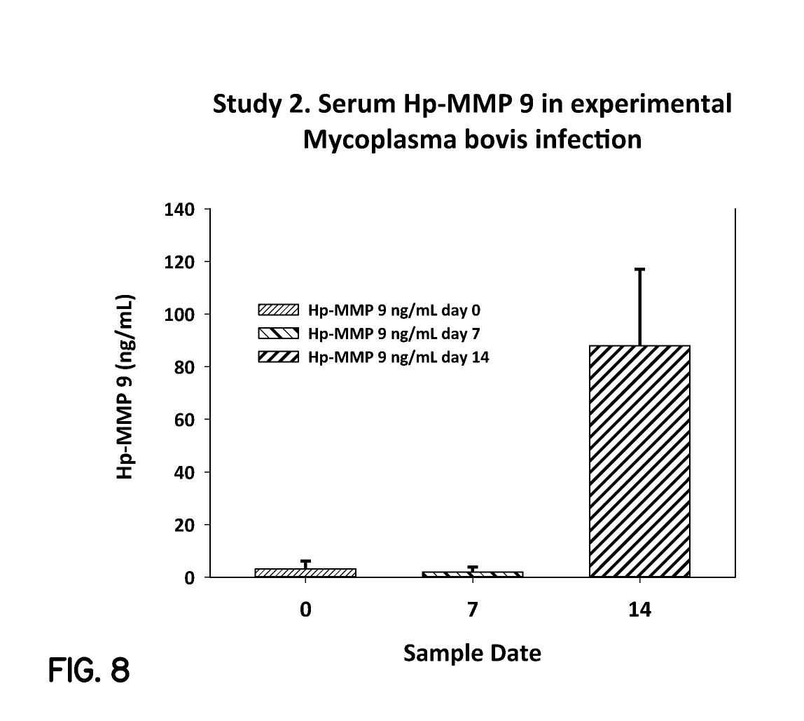

Serum samples were obtained from the laboratory of Dr. David Anderson (Kansas State University College of Veterinary Medicine, Manhattan, Kans.) to assay for serum markers of bovine respiratory disease. The serum was provided for Hp-MMP 9 complex assay associated with experimental Mycoplasma bovis inoculation. Animals (n=28) were bled immediately prior to (t=0) intra-tracheal instillation of 109 cfu viable M. bovis (n=24), or sham infused with sterile saline (n=4). All calves were then bled on days 7 and day 14. The ELISA test performed on the serum samples is as described above in Example 1, though those of ordinary skill in the art will recognize that parameters can be modified (and validated), such as is described below in Example 6. Serum Hp-MMP 9 complexes were absent in 26/28 animals prior to the start of the study. One animal with detectable serum Hp-MMP 9 also had elevated serum cardiac troponin I at the start of the study suggesting thoracic inflammation. The other animal had elevated serum haptoglobin on day 14 suggestive of chronic, non-specific inflammatory abnormalities. Serum Hp-MMP 9 complexes were detected in 4/28 animals on day 7 and 18/28 animals on day 14 (See FIG. 8).

### Example 4

This Example researches serum Hp-MMP 9 complexes in human patients with auto-immune diseases in comparison to osteoarthritis.

Serum samples from 29 subjects were obtained from the laboratory of Dr. Michael Wiseman (Cedar Sinai Medical Center in Southern California) for analysis of serum Hp-MMP 9 complexes. The ELISA test performed on the serum samples is as described above in Example 1, though those of ordinary skill in the art will recognize that parameters can be modified (and validated), such as is described below in Example 6. These samples (n=29) were from 3 different patient groups whose identity, disease and disease duration was not available to the individuals performing the ELISA studies on these samples. The samples from Patient Group #1 (n=9) were from patients with chronic osteoarthritis (“OA”); the samples from Patient Group #2 (n=10) were from patients with systemic lupus erythematosis (“SLE”); and the samples from Patient Group #3 (n=10) were from patients with rheumatoid arthritis (“RA”). The serum Hp-MMP 9 ELISA assay was performed on these samples blinded and the data were returned to Cedar Sinai Medical Center. The ELISA test performed on the serum samples is as described above in Example 1, though those of ordinary skill in the art will recognize that parameters can be modified (and validated), such as is described below in Example 6. After return of the data, Dr. Wiseman then provided the disease process associated with the tube number so that the disease processes were matched to serum Hp-MMP 9 concentrations.

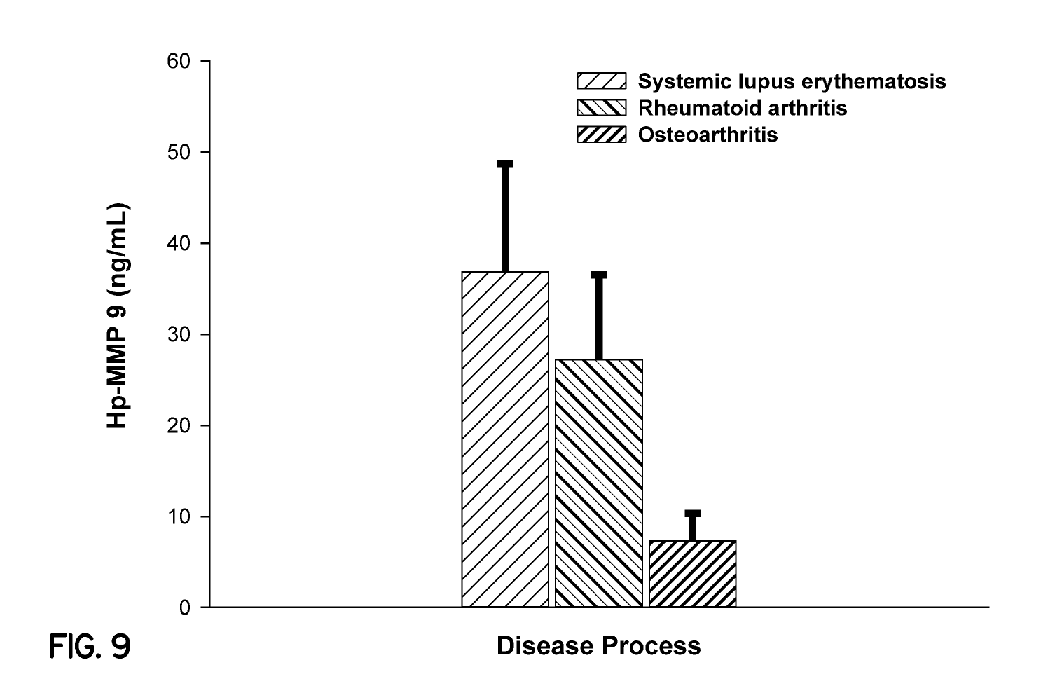

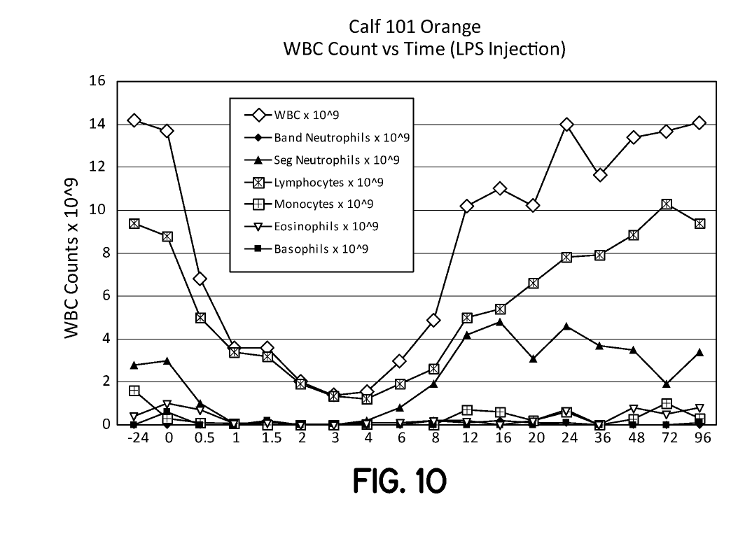

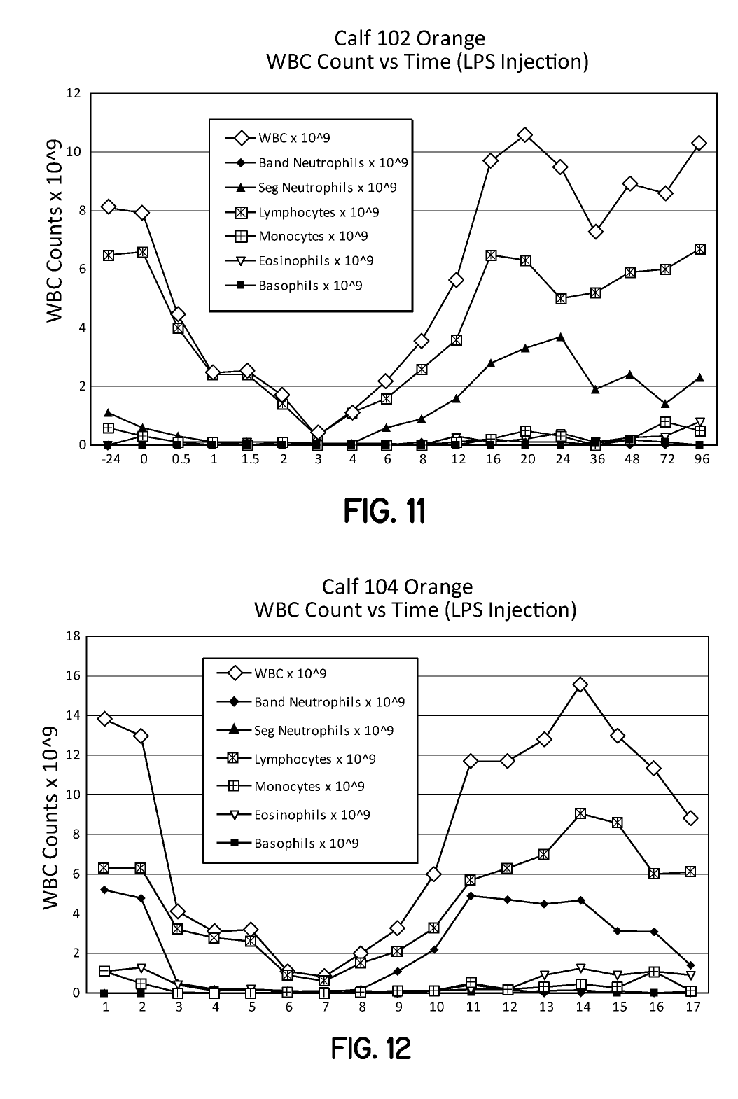

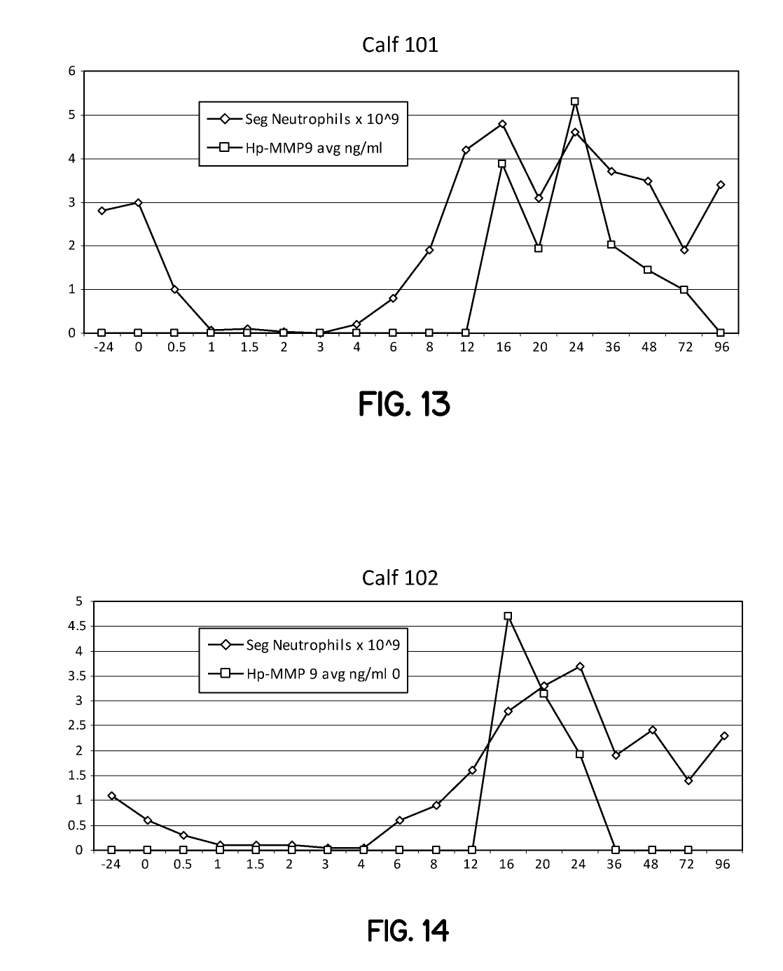

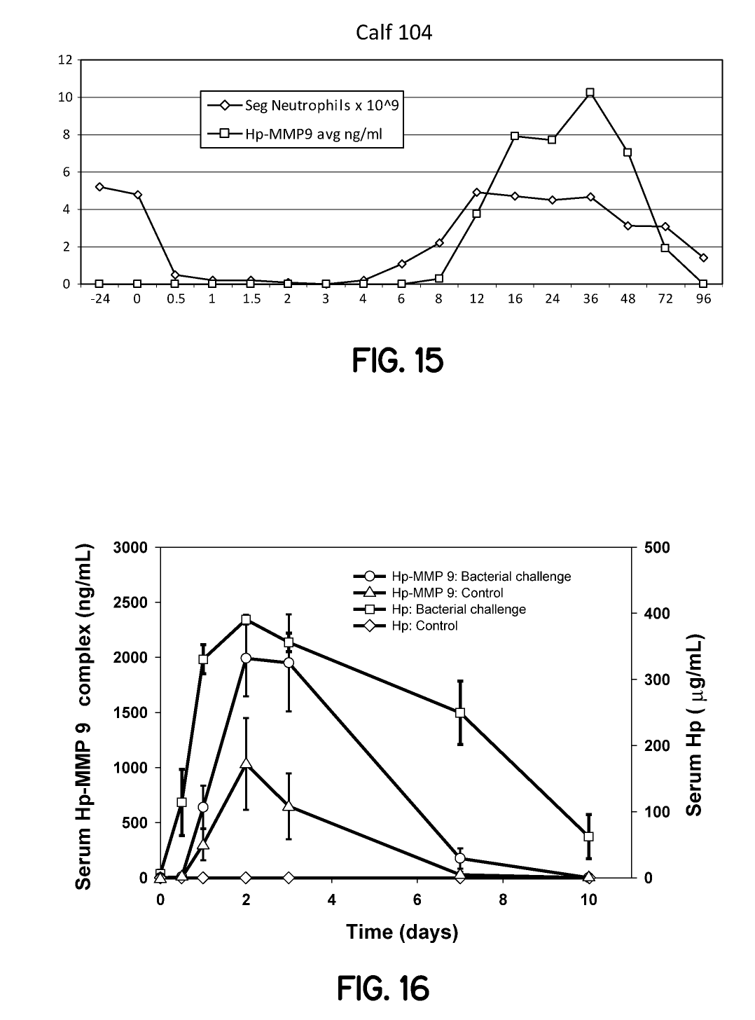

Differences in serum Hp-MMP 9 concentrations between SLE and OA (p=0.068) patients and the RA and OA patients (p=0.068) trended towards significance, suggesting active neutrophil mediated inflammation in the Hp-MMP 9 SLE and RA patients as compared to OA patients. Thus, serum Hp-MMP 9 appears to be present in humans afflicted with diseases characterized by neutophil activation (FIG. 9). Further, the historical clinical data of these patients (initial diagnosis, undergoing treatment, clinical remission) can be obtained in order to further elucidate the significance of this data.

### Example 5

This Example shows that experimental bacterial lung infection is associated with serum Hp-MMP 9 complexes. As is known to those of ordinary skill in the art, certain bacteria are some of the most prevalent organisms that cause bovine respiratory disease.

18 calves from Kansas State University College of Veterinary Medicine, Manhattan, Kans. were used in this Example. 10 of the 18 calves were inoculated with bacteria, and the remaining 8 calves were used as controls. The bacteria were administered to the 10 test calves via intra-pulmonary innoculation with live bacteria placed into the right cranio-ventral lung lobe (the predisposed lung lobe of the calf).

Serum samples were then obtained from each calf sequentially post-innoculation (time points for obtaining samples were Time=0 day, 0.5 day, 1 day, 2 days, 3 days, 7 days, and 10 days) by standard techniques that are known to those of ordinary skill in the relevant art (e.g., serum was harvested in routine fashion by centrifugation after clotting and frozen at −20° C. until analyzed).

ELISA for Hp-MMP 9 and Hp were then run based on the methods for these ELISA that are described above in Example 1. Data showing the effect of the bacteria on serum Hp-MMP 9 complexes and Hp in the calves is shown in FIG. 16. Following sample dilution according to that used in the M. bovis analysis, results were obtained without titrating the samples multiple times, which allows for a more rapid assay with the use of less blood sample form the subject animal.

Referring now to FIG. 16, and unlike the IV LPS challenge data that shows systemic response (as above in Examples 2 and 3), there is virtually no systemic Haptoglobin response in the control calves ( 4/8 calves had apparently been infected when housed with challenged calves as evidenced by serum Hp-MMP 9). These 8 exposed controls had 0 serum Hp responses in all cases, thereby distinguishing between these two tests (i.e., the Hp-MMP 9 ELISA and the Hp ELISA). As can be seen in FIG. 16, the Hp-MMP 9 response is detected at 24 hours and is present for up to 72 hours in high concentrations, with decline after that.

While not being bound to any theory, it is believed that this (0 serum Hp responses) is due to difference in the sensitivity of Haptoglobin ELISA and routine serum concentrations of Hp seen in sick cattle. The magnitude of the serum Hp is much lower in complex with MMP 9. Thus, IV-administered LPS activates neutrophils systemically (including liver) and both Hp-MMP 9 complexes are seen (as described in previous Examples). And, intra-bronchial bacterial LPS results in activation of neutrophils in lung and recruits them into lung (in this Example), but there is no systemic response as we saw with LPS.

### Example 6

This Example is directed to further methods for modifying and validating of the Hp-MMP 9 ELISA described herein.

The Examples described above were conducted utilizing the ELISA which was validated with affinity purified capture agent (mAb 10.1; anti-bovine MMP 9), affinity purified detection agent (rabbit, anti-bovine Hp-HRP conjugate) and purified Hp-MMP 9 complexes, as described in Example 1. These studies were validated as described in Bannikov, G A et al., 2011; Vet. Immunol. Immunopath 139:41-49, incorporated by reference herein in its entirety. Since this time, the focus has been on the use of serum samples from septic cows, analyzed as part of the previous study for standards (Bannikov et al., 2011, incorporated by reference herein in its entirety). Serum with known Hp-MMP 9 concentrations is currently being used to optimize ELISA conditions. Serum samples (serial dilutions) used as calibration curve were prepared and checkerboard titrations conducted to evaluate coating antibody concentration (2 ug/well to 64 ug/well) and detection antibody (1:5,000 to 1:160,000 fold dilutions). This demonstrated that coating antibody concentrations >2 ug/well are sufficient and that the standard curves are linear from 1:5K (0.2 ng/mL) to 1:160K (0.0063 ng/mL) detection antibody. However, as the concentration of secondary antibody declines, the slope of the line and dynamic range of calibration curve decreases from 1:5K to 1:160K. Further, evaluation of coating, washing and blocking buffers and detergent concentrations determined that BSA incorporating up to 5% tween-20 reduces non-specific background and increases dynamic range of assay to >1 absorbance units from bottom to top of calibration curve. The relative standard deviation for positive control calibrators was 0.6% (114 ng/mL) to 4.7% (14 ng/mL).

The above-described Examples demonstrate the relevance of the assay disclosed herein to known infectious agents and inflammatory challenges. Using challenge studies provides knowledge of sampling time points relative to infection/challenge. These responses can be used to define the most appropriate sampling times for use in field studies. From the LPS study, it appears that samples drawn between 0-4 days of arrival may be useful. From the M. bovis study, sampling times between 0-7 days may be useful. This information is useful in defining the utility of the Hp-MMP 9 test in field investigations of BRD, such as those proposed herein. Further, the method characterization studies have demonstrated the assay is robust and could be adapted to field use. Producing a synthetic source of Hp-MMP 9 should improve the ability to successfully implement a commercial, patient-side test for sub-clinical BRD in cattle.

The embodiments of the present invention recited herein are intended to be merely exemplary and those skilled in the art will be able to make numerous variations and modifications to it without departing from the spirit of the present invention. For example, while the Example described above discloses an ELISA and complex concentration ranges useful to test for an acute inflammatory condition in cattle, it will be recognized by those skilled in the art that such an ELISA can be adapted for testing for Hp-MMP 9 complexes in other mammals, such as a human, and the methods described herein can determine useful complex concentration ranges indicative of an acute inflammatory condition in such other mammal without undue experimentation. Moreover, Hp-MMP 9 complex ELISA is potentially useful in the diagnosis of other conditions, differing from acute sepsis, but associated with neutrophil degranulation. These conditions may include various types of arthritis, atherosclerosis, coronary plaque formation, etc. Notwithstanding the above, certain variations and modifications, while producing less than optimal results, may still produce satisfactory results. All such variations and modifications are intended to be within the scope of the present invention.

What is claimed is:

1. A method for detecting a haptoglobin-matrix metalloproteinase 9 (Hp-MMP 9) complex in a biological sample, comprising:
incubating a biological sample with a capture reagent immobilized on a solid support to bind Hp-MMP 9 to the capture reagent, wherein the capture reagent includes a monoclonal antibody that binds MMP9; and
detecting Hp-MMP 9 bound to the immobilized capture reagent by contacting the bound Hp-MMP 9 with a detectable antibody that binds to Hp.

2. The method of claim 1, further comprising determining the concentration of Hp-MMP 9 in the biological sample.

3. The method of claim 2, wherein determining the concentration of Hp-MMP 9 further comprises comparing the amount of detectable antibody bound to Hp-MMP 9 with a set of standards.

4. The method of claim 2, further comprising diagnosing an acute inflammatory condition or risk of an acute inflammatory condition by determining if the concentration of Hp-MMP 9 in the biological sample is within a range of concentrations indicative of an acute inflammatory condition or risk of an acute inflammatory condition.

5. The method of claim 4, wherein the range of concentration is about 900 ng/ml-about 1,500 ng/ml Hp-MMP 9.

6. The method of claim 1, wherein the biological sample is obtained from a mammal.

7. The method of claim 6, wherein the mammal is chosen from a human and cattle.

8. The method of claim 5, wherein the biological sample is obtained from cattle.

9. The method of claim 1, wherein the detectable antibody that binds to Hp includes a detection agent chosen from a chromogenic detection agent, a fluorogenic detection agent, an enzymatic detection agent, and an electrochemiluminescent detection agent.

10. The method of claim 9, wherein the detection agent is horseradish peroxidase (HRP).

11. The method of claim 10, wherein the detectable antibody is HRP-conjugated rabbit anti-bovine Hp.

12. A process for identifying a patient with an acute inflammatory condition or at risk of an acute inflammatory condition by determining concentration of Hp-MMP 9 in a bodily fluid sample, comprising:
(a) obtaining first monoclonal antibodies specific for one of MMP9 or Hp and attaching said monoclonal antibodies to a solid support;
(b) obtaining a sample of a bodily fluid from a patient wherein said sample is suspected of containing Hp-MMP 9 or immunogenic fragments of Hp-MMP 9;
(c) adding said sample to said monoclonal antibodies of step (a) wherein said Hp-MMP 9 or immunogenic fragments of Hp-MMP 9 contained in said sample is captured by said monoclonal antibodies;
(d) providing second antibodies specific for the other of MMP9 and Hp;
(e) labeling said second antibodies with a detector;
(f) adding said second antibodies of step (e) to said Hp-MMP 9 or immunogenic fragments of Hp-MMP 9 captured in step (c) wherein said second antibodies of step (e) bind to said Hp-MMP 9 or immunogenic fragments of Hp-MMP 9 captured in step (c);
(g) adding a reporter that reacts with said detector to form a reaction product; and
(h) measuring said reaction product to determine concentration of Hp-MMP 9 in said sample; and
(i) determining if said concentration of Hp-MMP 9 in said sample is elevated above a selected cut-off concentration indicative of concentration of Hp-MMP 9, wherein said elevated concentration identifies that the patient has an acute inflammatory condition or is at risk of an acute inflammatory condition.

13. The process of claim 12, wherein said cut-off concentration is about 900 ng/ml.

14. The process of claim 12, wherein the detector is horseradish peroxidase.

15. The process of claim 14, wherein the reporter is hydrogen peroxide.

16. A method for detecting Hp-MMP 9 in a biological sample comprising the steps of:
(a) providing polyclonal or monoclonal antibodies against MMP9;
(b) providing a microtiter plate coated with the antibodies;
(c) adding the biological sample to the microtiter plate;
(d) providing horseradish peroxidase-anti-Hp conjugates reactive with Hp to the microtiter plate;
(e) providing hydrogen peroxide to the microtiter plate; and
(f) comparing the reaction which occurs as a result of steps (a) to (e) with a standard curve to determine the level of Hp-MMP 9.

17. The method of claim 16, further comprising diagnosing an acute inflammatory condition or risk or an acute inflammatory condition if the level of Hp-MMP 9 in the biological sample is within a range of concentration indicative of an acute inflammatory condition or risk or an acute inflammatory condition.

18. The method of claim 17, wherein the range of concentration is about 900 ng/ml-about 1,500 ng/ml Hp-MMP 9.

19. The method of claim 16, wherein the biological sample is obtained from a mammal.

20. The method of claim 19, wherein the mammal is chosen from a human and cattle.

21. The method of claim 18, wherein the biological sample is obtained from cattle.
